SEQUENCE LISTING (27 sequences)

SEQ ID NO: 1 - RNA, 19 nt, Artificial Sequence
  siRNA_1
ccguaugcagcacaagaaa

SEQ ID NO: 2 - RNA, 19 nt, Artificial Sequence
  siRNA_2
guaugcagcacaagaaaga

SEQ ID NO: 3 - DNA, 89 bp, Artificial Sequence
  DRIVE_PCR_Rev_Primer_1
caagcagaagacggcatacgagatcactgtgtgactggagttcagacgtgtgctcttccgatctgaggttcagagttctacagtccgaa

SEQ ID NO: 4 - DNA, 89 bp, Artificial Sequence
  DRIVE_PCR_Rev_Primer_2
caagcagaagacggcatacgagatattggcgtgactggagttcagacgtgtgctcttccgatctgaggttcagagttctacagtccgaa

SEQ ID NO: 5 - DNA, 89 bp, Artificial Sequence
  DRIVE_PCR_Rev_Primer_3
caagcagaagacggcatacgagatgatctggtgactggagttcagacgtgtgctcttccgatctgaggttcagagttctacagtccgaa

SEQ ID NO: 6 - DNA, 89 bp, Artificial Sequence
  DRIVE_PCR_Rev_Primer_4
caagcagaagacggcatacgagattcaagtgtgactggagttcagacgtgtgctcttccgatctgaggttcagagttctacagtccgaa

SEQ ID NO: 7 - DNA, 89 bp, Artificial Sequence
  DRIVE_PCR_Rev_Primer_5
caagcagaagacggcatacgagatctgatcgtgactggagttcagacgtgtgctcttccgatctgaggttcagagttctacagtccgaa

SEQ ID NO: 8 - DNA, 89 bp, Artificial Sequence
  DRIVE_PCR_Rev_Primer_6
caagcagaagacggcatacgagataagctagtgactggagttcagacgtgtgctcttccgatctgaggttcagagttctacagtccgaa

SEQ ID NO: 9 - DNA, 89 bp, Artificial Sequence
  DRIVE_PCR_Rev_Primer_7
caagcagaagacggcatacgagatgtagccgtgactggagttcagacgtgtgctcttccgatctgaggttcagagttctacagtccgaa

SEQ ID NO: 10 - DNA, 89 bp, Artificial Sequence
  DRIVE_PCR_Rev_Primer_8
caagcagaagacggcatacgagattacaaggtgactggagttcagacgtgtgctcttccgatctgaggttcagagttctacagtccgaa

SEQ ID NO: 11 - DNA, 89 bp, Artificial Sequence
  DRIVE_PCR_Rev_Primer_9
caagcagaagacggcatacgagatttgactgtgactggagttcagacgtgtgctcttccgatctgaggttcagagttctacagtccgaa

SEQ ID NO: 12 - DNA, 89 bp, Artificial Sequence
  DRIVE_PCR_Rev_Primer_10
caagcagaagacggcatacgagatggaactgtgactggagttcagacgtgtgctcttccgatctgaggttcagagttctacagtccgaa

SEQ ID NO: 13 - DNA, 89 bp, Artificial Sequence
  DRIVE_PCR_Rev_Primer_11
caagcagaagacggcatacgagattgacatgtgactggagttcagacgtgtgctcttccgatctgaggttcagagttctacagtccgaa

SEQ ID NO: 14 - DNA, 89 bp, Artificial Sequence
  DRIVE_PCR_Rev_Primer_12
caagcagaagacggcatacgagatggacgggtgactggagttcagacgtgtgctcttccgatctgaggttcagagttctacagtccgaa

SEQ ID NO: 15 - DNA, 89 bp, Artificial Sequence
  DRIVE_PCR_Rev_Primer_13
caagcagaagacggcatacgagatctctacgtgactggagttcagacgtgtgctcttccgatctgaggttcagagttctacagtccgaa

SEQ ID NO: 16 - DNA, 89 bp, Artificial Sequence
  DRIVE_PCR_Rev_Primer_14
caagcagaagacggcatacgagatgcggacgtgactggagttcagacgtgtgctcttccgatctgaggttcagagttctacagtccgaa

SEQ ID NO: 17 - DNA, 89 bp, Artificial Sequence
  DRIVE_PCR_Rev_Primer_15
caagcagaagacggcatacgagattttcacgtgactggagttcagacgtgtgctcttccgatctgaggttcagagttctacagtccgaa

SEQ ID NO: 18 - DNA, 89 bp, Artificial Sequence
  DRIVE_PCR_Rev_Primer_16
caagcagaagacggcatacgagatggccacgtgactggagttcagacgtgtgctcttccgatctgaggttcagagttctacagtccgaa

SEQ ID NO: 19 - DNA, 89 bp, Artificial Sequence
  DRIVE_PCR_Rev_Primer_17
caagcagaagacggcatacgagatcgaaacgtgactggagttcagacgtgtgctcttccgatctgaggttcagagttctacagtccgaa

SEQ ID NO: 20 - DNA, 89 bp, Artificial Sequence
  DRIVE_PCR_Rev_Primer_18
caagcagaagacggcatacgagatcgtacggtgactggagttcagacgtgtgctcttccgatctgaggttcagagttctacagtccgaa

SEQ ID NO: 21 - DNA, 89 bp, Artificial Sequence
  DRIVE_PCR_Rev_Primer_19
caagcagaagacggcatacgagatccactcgtgactggagttcagacgtgtgctcttccgatctgaggttcagagttctacagtccgaa

SEQ ID NO: 22 - DNA, 89 bp, Artificial Sequence
  DRIVE_PCR_Rev_Primer_20
caagcagaagacggcatacgagatatcagtgtgactggagttcagacgtgtgctcttccgatctgaggttcagagttctacagtccgaa

SEQ ID NO: 23 - DNA, 89 bp, Artificial Sequence
  DRIVE_PCR_Rev_Primer_21
caagcagaagacggcatacgagataggaatgtgactggagttcagacgtgtgctcttccgatctgaggttcagagttctacagtccgaa

SEQ ID NO: 24 - DNA, 89 bp, Artificial Sequence
  DRIVE_PCR_Rev_Primer_22
caagcagaagacggcatacgagatcttttggtgactggagttcagacgtgtgctcttccgatctgaggttcagagttctacagtccgaa

SEQ ID NO: 25 - DNA, 89 bp, Artificial Sequence
  DRIVE_PCR_Rev_Primer_23
caagcagaagacggcatacgagattagttggtgactggagttcagacgtgtgctcttccgatctgaggttcagagttctacagtccgaa

SEQ ID NO: 26 - DNA, 89 bp, Artificial Sequence
  DRIVE_PCR_Rev_Primer_24
caagcagaagacggcatacgagatccggtggtgactggagttcagacgtgtgctcttccgatctgaggttcagagttctacagtccgaa

SEQ ID NO: 27 - DNA, 51 bp, Artificial Sequence
  DRIVE_PCR_Fwd_Universal_Primer
aatgatacggcgaccaccgagatctacacattcgcaccagcacgctacgca

(19) United States
(12) Patent Application Publication
(10) Pub. No.: US 2021/0348238 A1
(43) Pub. Date: Nov. 11, 2021

(54) TUMOR MUTATION BURDEN ALONE OR IN COMBINATION WITH IMMUNE MARKERS AS BIOMARKERS FOR PREDICTING RESPONSE TO TARGETED THERAPY

(71) Applicant: Novartis AG, Basel (CH)

(72) Inventors: Jan Christoph BRASE, Freiburg im breisgau (DE); James GARRETT, Cambridge, MA (US); Catarina CAMPBELL, Cambridge, MA (US)

(21) Appl. No.: 17/285,312

(22) PCT Filed: Oct. 15, 2019
(86) PCT No.: PCT/IB2019/058770

§ 371 Date: Apr. 14, 2021

Related U.S. Application Data
(60) Provisional application No. 62/747,730, filed on Oct. 19, 2018.
(60) Provisional application No. 62/746,227, filed on Oct. 16, 2018.

(51) Int. Cl. C12Q 1/6886

(52) U.S. Cl. CPC C12Q 1/6886; C12Q 2600/158; C12Q 2600/156; C12Q 2600/106

(57) ABSTRACT

The invention relates to the use of biomarkers for predicting the response to cancer (e.g. melanoma) treatments, for selecting a treatment for a cancer patient (e.g. using targeted therapy, e.g. using a BRAF and/or MEK inhibitor), for stratifying cancer patients into different treatment groups, for treating cancer patients, and for predicting clinical outcome in cancer.

### FIELD OF THE INVENTION

The invention relates to the use of biomarkers for predicting the response to cancer (e.g. melanoma) treatments, for selecting a treatment for a cancer patient (e.g. using targeted therapy, e.g. using a BRAF and/or MEK inhibitor), for stratifying cancer patients into different treatment groups, for treating cancer patients, and for predicting clinical outcome in cancer.

### BACKGROUND OF THE INVENTION

Cancer is a leading cause of death worldwide, accounting for over 8 million deaths per year. Melanoma is a malignant tumor that arises from uncontrolled proliferation of pigment-producing cells. Melanoma is one of the most common cancers, its prevalence is rising, and the majority of skin cancer deaths result from melanoma.

Apart from traditional cancer therapies as chemotherapy and radiation, new strategies have been developed to treat cancer. Among those are targeted therapies and immuno-oncology therapies. Targeted therapies include small molecule inhibitors and tumor-targeted antibodies that act by inhibiting drivers of growth that are specific to the tumor. Immuno-oncology therapies are based on the concept of activating the patient's immune system to produce antitumor immunity. Checkpoint inhibition is an immuno-oncology therapy that acts on inhibitory signaling pathways whose function is to suppress T cells. When this suppression is removed by checkpoint inhibition, this can unleash antitumor T cell activity.

Finding the right therapy for a cancer patient with melanoma (stage I-IV) is critical because a cancer can grow and metastasize extremely quickly and surgical resection alone may not be sufficient for cure due to the presence of undetected disseminated tumor cells. Targeted therapy and immuno-oncology therapy are both well established in advanced melanoma (stage IV) and were recently also approved in early stage Ill melanoma (adjuvant setting). It is important to identify responders to immuno-oncology and targeted therapy, as both treatment strategies are valuable treatment options in early and advanced melanoma

Tumor mutation burden (TMB) is a genomic biomarker, which measures the number of somatic mutations in the coding area of a tumor genome. The level of TMB was recently found to be associated with response to immuno-oncology therapy, and may be used to predict response to immuno-oncology therapy. The basis for the association between TMB level and response to immuno-oncology therapy is that a high TMB level increases the likelihood that immunogenic non-synonymous mutations are present, which can be cross-presented as neoantigens to immune cells, which eventually leads to an activation of the immune system. After unleashing the suppressed T-cell activity with checkpoint inhibitors, there is an increased likelihood for a sustainable immune response for tumors with high tumor mutation burden and a good rationale for TMB as a predictive marker for 10 therapy response. Methods for measuring the TMB level have been disclosed in WO 2018/068028 and are hereby incorporated by reference in their entirety. For targeted therapy (e.g. BRAF and/or MEK inhibitor in melanoma), there are no established predictive markers and TMB has not been analyzed in patients treated with a BRAF and/or MEK inhibitor in the adjuvant setting.

### SUMMARY OF THE INVENTION

In accordance with the present invention, it has now been discovered that cancer patients such as e.g. melanoma patients with a low TMB level have a greater benefit from a targeted therapy. This is the opposite of what was known about TMB and immuno-oncology therapy, where typically a high TMB level can be used to predict a benefit from an immuno-oncology therapy.

The present invention provides TMB as a biomarker for predicting the response to cancer treatments, e.g. to a melanoma treatment, for selecting a treatment for a cancer patient, e.g. a melanoma patient, for stratifying cancer patients, e.g. melanoma patients, into different treatment groups, for treating cancer patients, e.g. melanoma patients, and for predicting clinical outcome, e.g. melanomas.

In one aspect, the invention provides a method of identifying a melanoma patient who may benefit from a targeted therapy comprising an agent targeting BRAF and/or MEK; the method comprising scoring the tumor mutation burden (TMB) in a sample from the patient, wherein a low TMB score identifies the patient as one who may benefit from a targeted therapy comprising an agent targeting BRAF and/or MEK.

In another aspect, the invention provides a method of selecting a therapy for a melanoma patient; the method comprising scoring the TMB in a sample from the patient, wherein a low TMB score identifies the patient as one who may benefit from a targeted therapy comprising an agent targeting BRAF and/or MEK.

In another aspect, the invention provides a method of treating a melanoma patient, the method comprising (a) scoring the TMB in a sample from the patient, wherein the TMB score is low, and (b) administering an effective amount of targeted therapy comprising an agent targeting BRAF and/or MEK to the patient.

In another aspect, the invention provides a method of treating a melanoma patient, the method comprising administering to the patient an effective amount of a targeted therapy comprising an agent targeting BRAF and/or MEK, wherein prior to the administration a low TMB score has been determined from a sample from the patient.

In another aspect, the invention provides a method of treating a melanoma patient with a targeted therapy comprising an agent targeting BRAF and/or MEK, wherein the melanoma of said patient is characterized as having a low TMB score.

In another aspect, the invention provides the use of TMB as a predictive marker for selecting melanoma patients for treatment with a targeted therapy comprising an agent targeting BRAF and/or MEK, wherein a patient is treated with said treatment if the sample of the patient is determined as having a low TMB score.

In another aspect, the invention provides a method of identifying a melanoma patient who may benefit from a targeted therapy comprising an agent targeting BRAF and/or MEK; the method comprising scoring the i) TMB and ii) immune activation in a sample from the patient, wherein a high TMB score with a high immune activation score identifies the patient as one who may benefit from a targeted therapy comprising an agent targeting BRAF and/or MEK.

In another aspect, the invention provides a method of selecting a therapy for a melanoma patient; the method comprising scoring the i) TMB and ii) immune activation level in a sample from the patient, wherein a high TMB score with a high immune activation score identifies the patient as one who may benefit from a targeted therapy comprising an agent targeting BRAF and/or MEK.

In another aspect, the invention provides a method of treating a melanoma patient, the method comprising (a) scoring the i) TMB and ii) immune activation level in a sample from the patient, wherein the both the TMB and the immune activation scores are high, and (b) administering an effective amount of a targeted therapy comprising an agent targeting BRAF and/or MEK to the patient.

In another aspect, the invention provides a method of treating a melanoma patient, the method comprising administering to the patient an effective amount of a targeted therapy comprising an agent targeting BRAF and/or MEK, wherein prior to the administration a high TMB score with a high immune activation score has been determined from a sample from the patient.

In another aspect, the invention provides a method of treating a patient having melanoma with a therapy comprising an agent targeting BRAF and/or MEK, wherein the melanoma of the patient is characterized as having a high TMB score with a high immune activation score.

In another aspect, the invention provides the use of TMB and immune activation as a predictive marker for selecting melanoma patients for treatment with a therapy comprising an agent targeting BRAF and/or MEK, wherein a patient is treated with said therapy if the sample of the patient is determined as having a high TMB score with a high immune activation score.

In another aspect, the invention provides a method of stratifying melanoma patients into two groups, one that may benefit from a targeted therapy comprising an agent targeting BRAF and/or MEK, and another one that may benefit from an immuno-oncology therapy; the method comprising scoring the i) TMB and ii) immune activation level in a sample from the patient, wherein a patient who may benefit from a targeted therapy comprising an agent targeting BRAF and/or MEK has either a high TMB score with a high immune activation score or a low TMB score, irrespective of immune activation score, while a patient who may benefit from an immuno-oncology therapy has a high TMB score with a low immune activation score.

In another aspect, the invention provides a method of stratifying melanoma patients into two groups for selecting a therapy; the method comprising scoring i) the TMB and ii) immune activation level in a sample from the patient, wherein a high TMB score with a high immune activation score or a low TMB score, irrespective of immune activation score identifies a patient who may benefit from a targeted therapy comprising an agent targeting BRAF and/or MEK, while a high TMB score with a low immune activation score identifies the patient as one with less sustained response to targeted therapy. Published data sets in lung cancer suggest that these patients may benefit from an immuno-oncology therapy.

In another aspect, the invention provides a method of treating a melanoma patient, the method comprising (a) scoring the i) TMB and ii) immune activation level in a sample from the patient and (b) administering an effective amount of therapy to the patient, wherein the therapy is a targeted therapy comprising an agent targeting BRAF and/or MEK for patients with a high TMB score with a high immune activation score or a low TMB score, irrespective of immune activation score and the therapy is an immuno-oncology therapy for patients with a high TMB score with a low immune activation score.

In another aspect, the invention provides a method of treating a melanoma patient, the method comprising administering to the patient either (a) an effective amount of a targeted therapy comprising an agent targeting BRAF and/or MEK, wherein prior to the administration a high TMB score with a high immune activation score or a low TMB score, irrespective of immune activation score has been determined from a sample from the patient, or (b) an effective amount of an immuno-oncology therapy, wherein prior to the administration a high TMB score with a low immune activation score has been determined from a sample from the patient.

In another aspect, the invention provides a method of treating a patient having melanoma with either (a) a targeted therapy comprising an agent targeting BRAF and/or MEK, wherein the melanoma of said patient is characterized as having a high TMB score with a high immune activation score or a low TMB score, irrespective of immune activation score, or (b) an immuno-oncology therapy, wherein the melanoma of said patient is characterized as having a high TMB score with a low immune activation score.

In another aspect, the invention provides the use of TMB and immune activation as a predictive marker for selecting melanoma patients for treatment with either (a) a targeted therapy comprising an agent targeting BRAF and/or MEK, wherein the melanoma of said patient is characterized as having a high TMB score with a high immune activation score or a low TMB score, irrespective of immune activation score, or (b) an immuno-oncology therapy, wherein the melanoma of said patient is characterized as having a high TMB score with a low immune activation score.

In another aspect, the invention provides a method of stratifying melanoma patients into two groups, one that may benefit from a targeted therapy comprising an agent targeting BRAF and/or MEK, and second one that may benefit from a combination of a targeted therapy comprising an agent targeting BRAF and/or MEK and an immuno-oncology therapy; the method comprising scoring the TMB in a sample from the patient, wherein a patient who may benefit from a targeted therapy comprising an agent targeting BRAF and/or MEK has a low TMB score, while a patient who may benefit from a combination of a targeted therapy comprising an agent targeting BRAF and/or MEK and an immuno-oncology therapy has a high TMB score.

In another aspect, the invention provides a method of stratifying melanoma patients into two groups for selecting a therapy; the method comprising scoring the TMB in a sample from the patient, wherein a low TMB score identifies a patient who may benefit from a targeted therapy comprising an agent targeting BRAF and/or MEK, while high TMB score identifies the patient as one who may benefit from an combination of a targeted therapy comprising an agent targeting BRAF and/or MEK and an immuno-oncology therapy.

In another aspect, the invention provides a method of treating a melanoma patient, the method comprising (a) scoring the TMB in a sample from the patient and (b) administering an effective amount of therapy to the patient, wherein the therapy is a targeted therapy comprising an agent targeting BRAF and/or MEK for patients with low TMB score, and the therapy is a combination of a targeted therapy comprising an agent targeting BRAF and/or MEK and an immuno-oncology therapy for patients with a high TMB score.

In another aspect, the invention provides a method of treating a melanoma patient, the method comprising administering to the patient either (a) an effective amount of a targeted therapy comprising an agent targeting BRAF and/or MEK, wherein prior to the administration a low TMB score has been determined from a sample from the patient, or (b) an effective amount of a combination of a targeted therapy comprising an agent targeting BRAF and/or MEK and an immuno-oncology therapy, wherein prior to the administration a high TMB score has been determined from a sample from the patient.

In another aspect, the invention provides a method of treating a patient having melanoma with either (a) a targeted therapy comprising an agent targeting BRAF and/or MEK, wherein the melanoma of said patient is characterized as having a low TMB score, or (b) a combination of a targeted therapy comprising an agent targeting BRAF and/or MEK and an immuno-oncology therapy, wherein the melanoma of said patient is characterized as having a TMB score.

In another aspect, the invention provides the use of TMB as a predictive marker for selecting melanoma patients for treatment with a targeted therapy comprising a BRAF and/or MEK inhibitor in combination with an immuno-oncology treatment, wherein the targeted therapy comprises an agent targeting BRAF and/or MEK and the immuno-oncology treatment is a PD-1 or PD-L1 binding antagonist, comprising scoring the TMB in a sample from said patient.

In another aspect, the invention provides a method of stratifying melanoma patients into two groups, one that may benefit from a targeted therapy comprising an agent targeting BRAF and/or MEK, and second one that may benefit from a combination of a targeted therapy comprising an agent targeting BRAF and/or MEK and an immuno-oncology therapy; the method comprising scoring the i) TMB and ii) immune activation level in a sample from the patient, wherein a patient who may benefit from a targeted therapy comprising an agent targeting BRAF and/or MEK has a low TMB score with a low immune activation score, while a patient who may benefit from a combination of a targeted therapy comprising an agent targeting BRAF and/or MEK and an immuno-oncology therapy has either a low TMB score with a high immune activation score or a high TMB score, irrespective of immune activation score.

In another aspect, the invention provides a method of stratifying melanoma patients into two groups for selecting a therapy; the method comprising scoring i) the TMB and ii) immune activation level in a sample from the patient, wherein a low TMB score with a low immune activation score identifies a patient who may benefit from a targeted therapy comprising an agent targeting BRAF and/or MEK, while either a low TMB score with a high immune activation score or a high TMB score, irrespective of immune activation score identifies the patient as one who may benefit from an combination of a targeted therapy comprising an agent targeting BRAF and/or MEK and an immuno-oncology therapy.

In another aspect, the invention provides a method of treating a melanoma patient, the method comprising (a) scoring the i) TMB and ii) immune activation level in a sample from the patient and (b) administering an effective amount of therapy to the patient, wherein the therapy is a targeted therapy comprising an agent targeting BRAF and/or MEK for patients with low TMB score with a low immune activation score, and the therapy is a combination of a targeted therapy comprising an agent targeting BRAF and/or MEK and an immuno-oncology therapy for patients with either a low TMB score with a high immune activation score or a high TMB score, irrespective of immune activation score.

In another aspect, the invention provides a method of treating a melanoma patient, the method comprising administering to the patient either (a) an effective amount of a targeted therapy comprising an agent targeting BRAF and/or MEK, wherein prior to the administration a low TMB score with a low immune activation score has been determined from a sample from the patient, or (b) an effective amount of a combination of a targeted therapy comprising an agent targeting BRAF and/or MEK and an immuno-oncology therapy, wherein prior to the administration either a low TMB score with a high immune activation score or a high TMB score, irrespective of immune activation score has been determined from a sample from the patient.

In another aspect, the invention provides a method of treating a patient having melanoma with either (a) a targeted therapy comprising an agent targeting BRAF and/or MEK, wherein the melanoma of said patient is characterized as having a low TMB score with a low immune activation score, or (b) an combination of a targeted therapy comprising an agent targeting BRAF and/or MEK and an immuno-oncology therapy, wherein the melanoma of said patient is characterized as having either a low TMB score with a high immune activation score or a high TMB score, irrespective of immune activation score.

In another aspect, the invention provides the use of TMB and immune activation as a predictive marker for selecting melanoma patients for treatment with a targeted therapy comprising a BRAF and/or MEK inhibitor in combination with an immuno-oncology treatment, wherein the targeted therapy comprises an agent targeting BRAF and/or MEK and the immuno-oncology treatment is a PD-1 or PD-L1 binding antagonist, comprising scoring the i) TMB and ii) immune activation level in a sample from said patient.

### BRIEF DESCRIPTION OF THE FIGURES

FIG. 1

TMB as detected by targeted sequencing in 368 patients of the Combi-AD trial. Different TMB cut-offs: median (a), 10 (b), and 16 (c) were used to classify patients into TMB-low and TMB-high subgroups. A greater treatment benefit was observed for patients classified as TMB-low. PBO=placebo, trt=treatment (Dabrafenib/Trametinib), RFS=relapse-free survival time.

FIG. 2

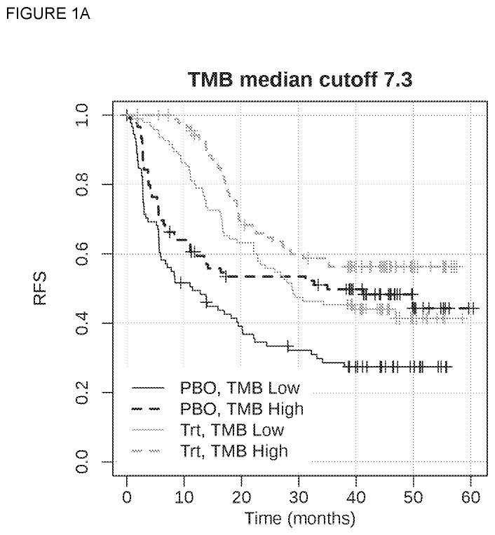

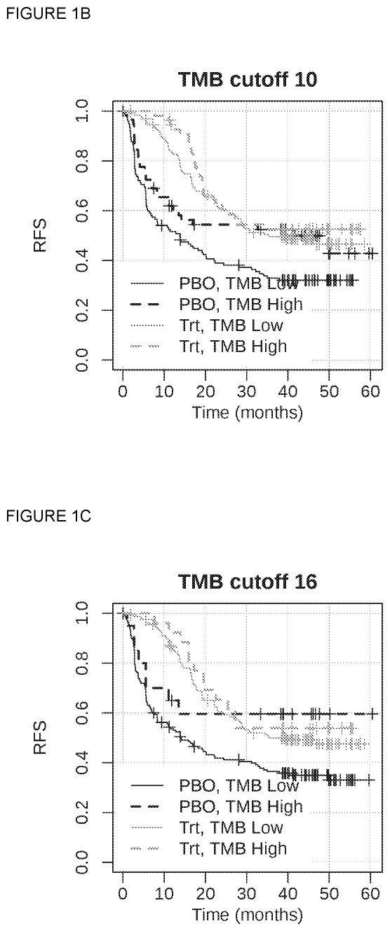

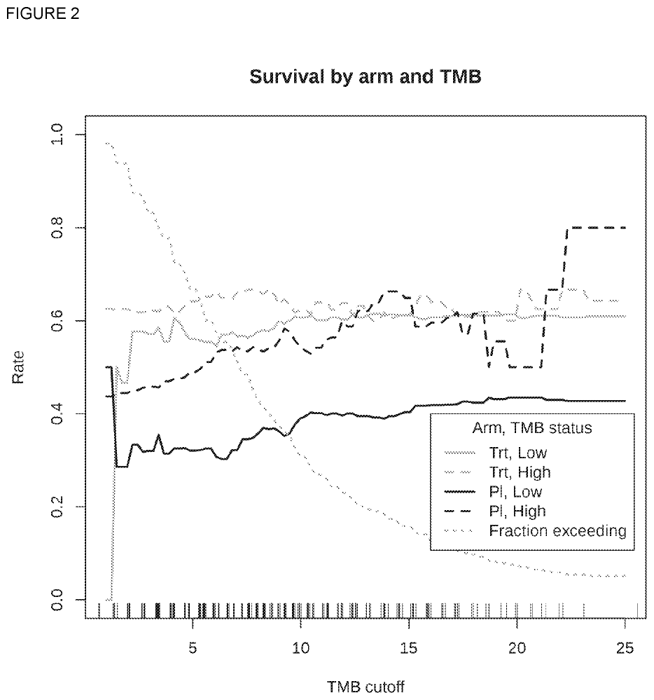

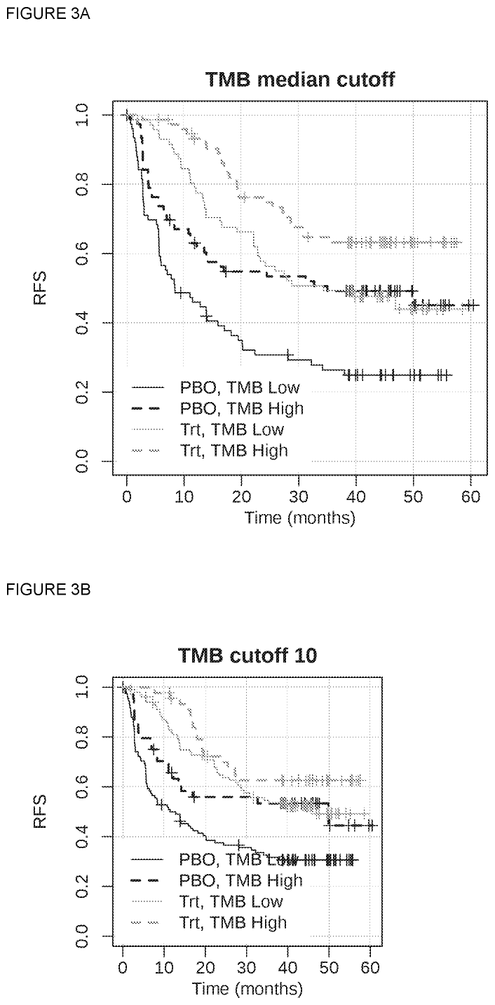

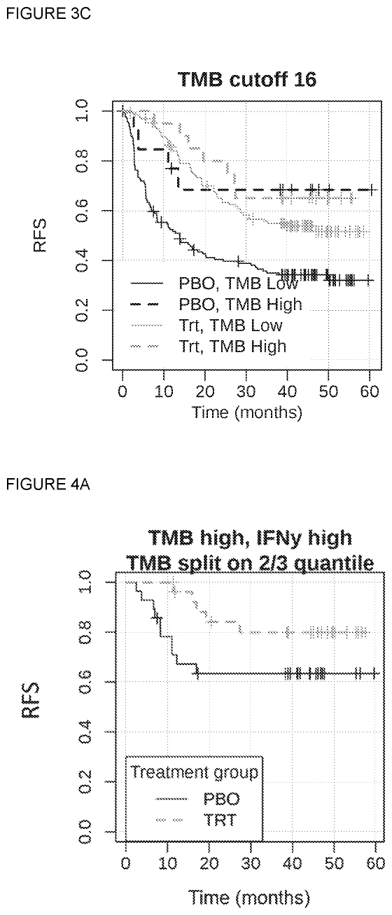

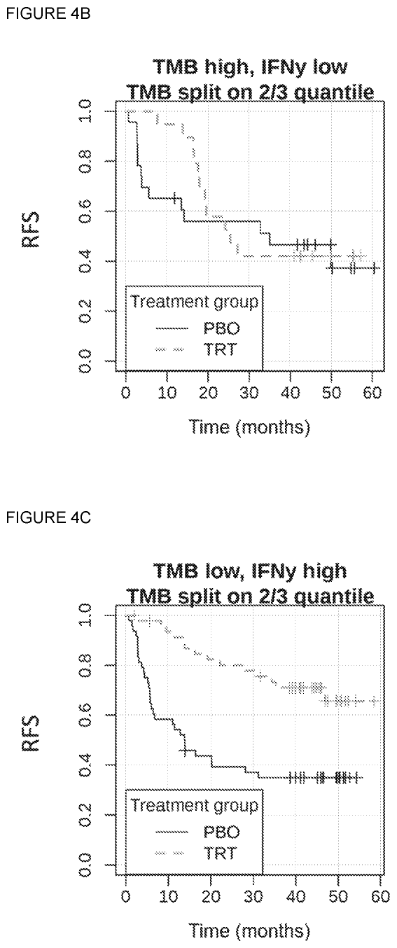

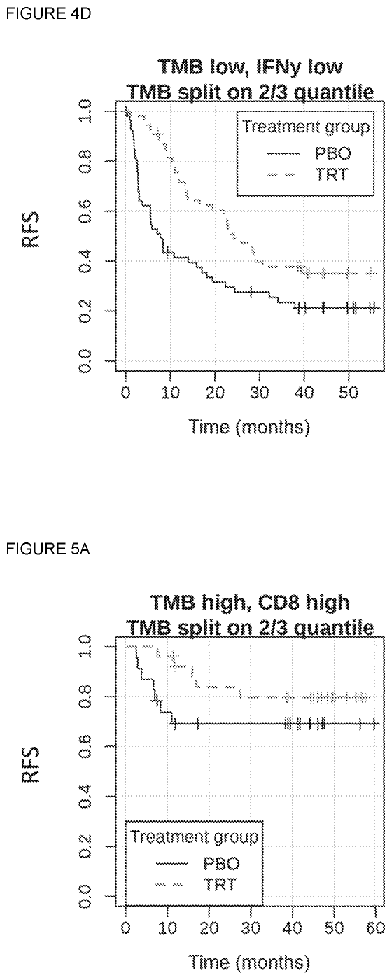

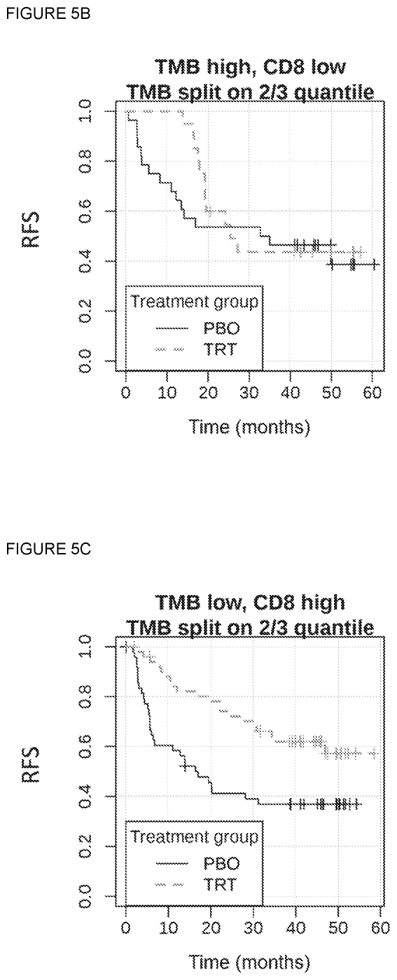

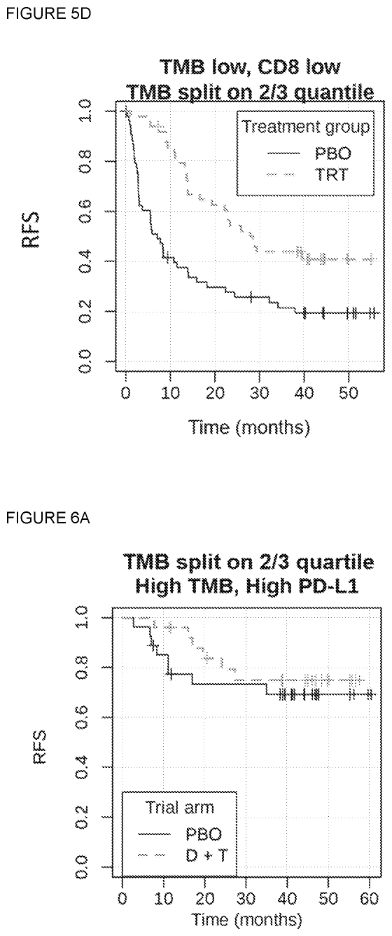

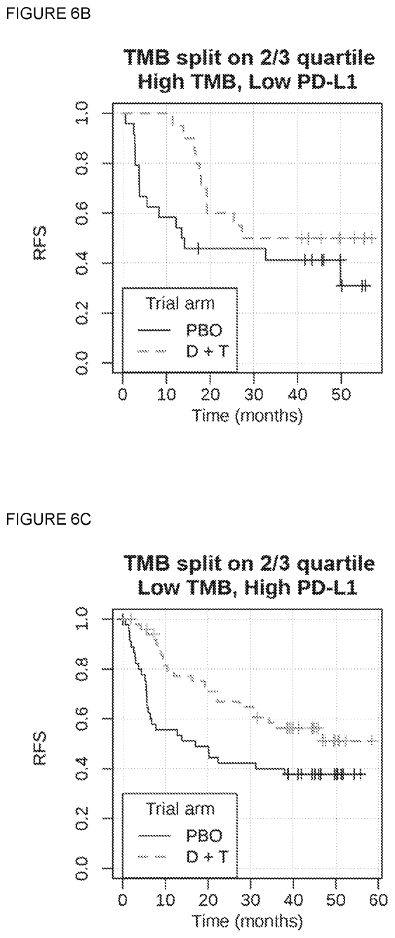

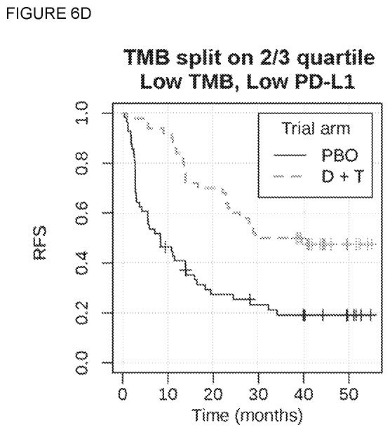

Different TMB cut-offs (x-axis) based on the available TMB data for 368 patients in comparison to the RFS rate at 24 month (y-axis) for the following subgroups of interest: TMB-low, Dabrafenib/Trametinib arm (solid grey curve), TMB-high, Dabrafenib/Trametinib arm (dotted grey curve), TMB-high, placebo arm (dotted black curve), TMB-low, placebo arm (solid black curve). Independently of the selected TMB cut-off, an excellent response to Dabrafenib and Trametinib is seen in the subgroup with low TMB levels (comparison of solid grey curve vs. solid black curve), whereas a less pronounced response is seen in patients with high TMB levels (comparison of dotted grey curve vs. dotted black curve).

FIG. 3

TMB as detected by targeted sequencing in 301 patients (DNA-seq and RNA data available) of the Combi-AD trial. Different TMB cut-offs: median (a), 10 (b), and 16 (c) were used to classify patients into TMB-low and TMB-high subgroups. A greater treatment benefit was observed for patients classified as TMB-low. PBO=placebo, trt=treatment (Dabrafenib/Trametinib). RFS=relapse-free survival time.

FIG. 4

TMB and IFN-γ gene expression signature results (Nanostring customized panel) in 301 patients (DNA-seq and RNA data available) of the Combi-AD trial. Patients were classified into TMB high (top third, ⅓ quantile) vs. TMB low and IFN-γ low vs. high (median split was used for the IFN-γ signature). Exploratory analysis of RFS in D+T vs Pbo arms in all TMB/IFN-γ subgroups suggested that low TMB or high TMB/high IFN-γ may be associated with greater RFS benefit than high TMB/low IFN-γ. PBO=placebo, trt=treatment (Dabrafenib/Trametinib). RFS=relapse-free survival time.

FIG. 5

TMB and T-cell/CD8 gene expression signature results (Nanostring customized panel) in 301 patients (DNA-seq and RNA data available) of the Combi-AD trial. Patients were classified into TMB high (top third, ⅓ quantile) vs. TMB low and T-cell/CD8 low vs. high (median split was used for the T-cell/CD8 signature). Exploratory analysis of RFS in D+T vs Pbo arms in all TMB/T-cell/CD8 subgroups suggested that low TMB or high TMB/high T-cell/CD8 may be associated with greater RFS benefit than high TMB/low T-cell/CD8. PBO=placebo, trt=treatment (Dabrafenib/Trametinib). RFS=relapse-free survival time.

FIG. 6

TMB and PD-L1 gene expression levels (Nanostring customized panel) in 301 patients (DNA-seq and RNA data available) of the Combi-AD trial. Patients were classified into TMB high (top third, ⅓ quantile) vs. TMB low and PD-L1 low vs. high (median split was used for the PD-L1 gene expression level). Exploratory analysis of RFS in D+T vs Pbo arms in all TMB/T-cell/CD8 subgroups suggested that low TMB or high TMB/high PD-L1 may be associated with greater RFS benefit than high TMB/low PD-L1. PBO=placebo, trt=treatment (Dabrafenib/Trametinib). RFS=relapse-free survival time

### DETAILED DESCRIPTION OF THE INVENTION

### Introduction

The present invention provides therapeutic and diagnostic methods and compositions for cancer, in particular melanoma. The invention is based, at least in part, on the discovery that determining the level of somatic mutations in a tumor and deriving a tumor mutation burden (TMB) score can be used as a biomarker (e.g. a predictive biomarker) in the treatment of a cancer patient, e.g. a melanoma patient, for diagnosing a cancer patient, for determining whether a cancer patient, e.g. a melanoma patient, is likely to respond to treatment with an cancer therapy that includes a targeted therapy, including agents targeting BRAF and/or MEK, for optimizing therapeutic efficacy of an cancer therapy, e.g. a melanoma therapy, that includes a targeted therapy including agents targeting BRAF and/or MEK, and for patient selection for an cancer therapy, e.g. a melanoma therapy, comprising a targeted therapy including agents targeting BRAF and/or MEK.

In one aspect, the invention provides a method of identifying a melanoma patient who may benefit from a targeted therapy comprising an agent targeting BRAF and/or MEK; the method comprising scoring the tumor mutation burden (TMB) in a sample from the patient, wherein a low TMB score identifies the patient as one who may benefit from a targeted therapy comprising an agent targeting BRAF and/or MEK.

Unless indicated otherwise, the term “agent targeting BRAF and/or MEK” preferably refers to a BRAF inhibitor and a MEK inhibitor, such as e.g. Dabrafenib and Trametinib or e.g. Vemurafenib and Cobimetinib. In one particular embodiment, the BRAF inhibitor is Dabrafenib and the MEK inhibitor is Trametinib.

Unless indicated otherwise, the term “melanoma” preferably refers to a BRAF V600 mutant melanoma. The melanoma can be a stage I, II, III, IV melanoma, preferably stage II, III or IV.

Unless indicated otherwise, the term “treatment” preferably refers to a first-, second-, third-, or fourth-line or beyond treatment in the advanced setting or to an adjuvant or neo-adjuvant treatment.

Unless indicated otherwise, a low TMB score is preferably 5 or less, 6 or less, 7 or less, 8 or less, 9 or less, 10 or less, 11 or less, 12 or less, 13 or less, 14 or less, 15 or less, or 16 or less mutations per megabase (mutations/Mb), more preferably 9 or less, 10 or less, or 11 or less mutations/Mb, more preferably 10 or less mutations/Mb or 11 or less mutations/Mb, and a high TMB score is preferably more than 5, more than 6, more than 7, more than 8, more than 9, more than 10, more than 11, more than 12, more than 13, more than 14, more than 15, or more than 16 mutations/Mb, more preferably more than 9, more than 10, or more than 11 mutations/Mb, more preferably more than 10 mutations/Mb or more than 11 mutations/Mb.

In another aspect, the invention provides a method of selecting a therapy for a melanoma patient; the method comprising scoring the TMB in a sample from the patient, wherein a low TMB score identifies the patient as one who may benefit from a targeted therapy comprising an agent targeting BRAF and/or MEK.

In some embodiments of any one of the two preceding aspects, the TMB score is low, and the method further comprises administering the patient an effective amount of a targeted therapy comprising an agent targeting BRAF and/or MEK.

In another aspect, the invention provides a method of treating a melanoma patient, the method comprising: (a) scoring the TMB in a sample from the patient, wherein the TMB score is low, and (b) administering an effective amount of targeted therapy comprising an agent targeting BRAF and/or MEK to the patient.

In another aspect, the invention provides a method of treating a melanoma patient, the method comprising administering to the patient an effective amount of a targeted therapy comprising an agent targeting BRAF and/or MEK, wherein prior to the administration a low TMB score has been determined from a sample from the patient.

In another aspect, the invention provides a method of treating a melanoma patient with a targeted therapy comprising an agent targeting BRAF and/or MEK, wherein the melanoma of said patient is characterized as having a low TMB score.

In another aspect, the invention provides the use of TMB as a predictive marker for selecting melanoma patients for treatment with a targeted therapy comprising an agent targeting BRAF and/or MEK, wherein a patient is treated with said treatment if the sample of the patient is determined as having a low TMB score.

In another aspect, the invention provides a method of identifying a melanoma patient who may benefit from a targeted therapy comprising an agent targeting BRAF and/or MEK; the method comprising scoring the i) TMB and ii) levels of immune activation level in a sample from the patient, wherein a high TMB score with a high immune activation score identifies the patient as one who may benefit from a targeted therapy comprising an agent targeting BRAF and/or MEK.

Unless indicated otherwise, the immune activation levels are preferably assessed by measuring tumor infiltrating lymphocytes, PD-L1, CD8, IFNy, or T-cell inflamed gene expression signatures.

In another aspect, the invention provides a method of selecting a therapy for a melanoma patient; the method comprising scoring the i) TMB and ii) immune activation level in a sample from the patient, wherein a high TMB score with a high immune activation score identifies the patient as one who may benefit from a targeted therapy comprising an agent targeting BRAF and/or MEK.

In some embodiments of any one of the preceding two aspects, both the TMB and the immune activation scores are high, and the method further comprises administering the patient an effective amount of a targeted therapy comprising an agent targeting BRAF and/or MEK.

In another aspect, the invention provides a method of treating a melanoma patient, the method comprising: (a) scoring the i) TMB and ii) immune activation level in a sample from the patient, wherein the both the TMB and the immune activation scores are high, and (b) administering an effective amount of a targeted therapy comprising an agent targeting BRAF and/or MEK to the patient.

In another aspect, the invention provides a method of treating a melanoma patient, the method comprising administering to the patient an effective amount of a targeted therapy comprising an agent targeting BRAF and/or MEK, wherein prior to the administration a high TMB score with a high immune activation score has been determined from a sample from the patient.

In another aspect, the invention provides a method of treating a patient having melanoma with a therapy comprising an agent targeting BRAF and/or MEK, wherein the melanoma of the patient is characterized as having a high TMB score with a high immune activation score.

In another aspect, the invention provides the use of TMB and immune activation as a predictive marker for selecting melanoma patients for treatment with a therapy comprising an agent targeting BRAF and/or MEK, wherein a patient is treated with said therapy if the sample of the patient is determined as having a high TMB score with a high immune activation score.

In another aspect, the invention provides a method of stratifying melanoma patients into two groups, one that may benefit from a targeted therapy comprising an agent targeting BRAF and/or MEK, and another one that may benefit from an immuno-oncology therapy; the method comprising scoring the i) TMB and ii) immune activation level in a sample from the patient, wherein a patient who may benefit from a targeted therapy comprising an agent targeting BRAF and/or MEK has either a high TMB score with a high immune activation score or a low TMB score, irrespective of immune activation score, while a patient who may benefit from an immuno-oncology therapy has a high TMB score with a low immune activation score.

Unless indicated otherwise, the term “the immuno-oncology therapy” preferably refers to a PD-1 or PDL-1 binding antagonist, either as single agents or in combination with another immuno-oncology therapy agent (e.g. anti-CTLA4).

In another aspect, the invention provides a method of stratifying melanoma patients into two groups for selecting a therapy; the method comprising scoring i) the TMB and ii) immune activation level in a sample from the patient, wherein a high TMB score with a high immune activation score or a low TMB score, irrespective of immune activation score identifies a patient who may benefit from a targeted therapy comprising an agent targeting BRAF and/or MEK, while a high TMB score with a low immune activation score identifies the patient as one who may benefit from an immuno-oncology therapy.

In some embodiments of any one of the preceding two aspects, either (a) a high TMB score with a high immune activation score or a low TMB score, irrespective of immune activation score has been determined and the method further comprises administering the patient an effective amount of a targeted therapy comprising an agent targeting BRAF and/or MEK, or (b) a high TMB score with a low immune activation score has been determined, and the method further comprises administering the patient an effective amount of immuno-oncology therapy.

In another aspect, the invention provides a method of treating a melanoma patient, the method comprising (a) scoring the i) TMB and ii) immune activation level in a sample from the patient and (b) administering an effective amount of therapy to the patient, wherein the therapy is a targeted therapy comprising an agent targeting BRAF and/or MEK for patients with a high TMB score with a high immune activation score or a low TMB score, irrespective of immune activation score and the therapy is an immuno-oncology therapy for patients with a high TMB score with a low immune activation score.

In another aspect, the invention provides a method of treating a melanoma patient, the method comprising administering to the patient either (a) an effective amount of a targeted therapy comprising an agent targeting BRAF and/or MEK, wherein prior to the administration a high TMB score with a high immune activation score or a low TMB score, irrespective of immune activation score has been determined from a sample from the patient, or (b) an effective amount of an immuno-oncology therapy, wherein prior to the administration a high TMB score with a low immune activation score has been determined from a sample from the patient.

In another aspect, the invention provides a method of treating a patient having melanoma with either (a) a targeted therapy comprising an agent targeting BRAF and/or MEK, wherein the melanoma of said patient is characterized as having a high TMB score with a high immune activation score or a low TMB score, irrespective of immune activation score, or (b) an immuno-oncology therapy, wherein the melanoma of said patient is characterized as having a high TMB score with a low immune activation score.

In another aspect, the invention provides the use of TMB and immune activation as a predictive marker for selecting melanoma patients for treatment with either (a) a targeted therapy comprising an agent targeting BRAF and/or MEK, wherein the melanoma of said patient is characterized as having a high TMB score with a high immune activation score or a low TMB score, irrespective of immune activation score, or (b) an immuno-oncology therapy, wherein the melanoma of said patient is characterized as having a high TMB score with a low immune activation score.

In another aspect, the invention provides a method of stratifying melanoma patients into two groups, one that may benefit from a targeted therapy comprising an agent targeting BRAF and/or MEK, and second one that may benefit from a combination of a targeted therapy comprising an agent targeting BRAF and/or MEK and an immuno-oncology therapy; the method comprising scoring the TMB in a sample from the patient, wherein a patient who may benefit from a targeted therapy comprising an agent targeting BRAF and/or MEK has a low TMB score, while a patient who may benefit from a combination of a targeted therapy comprising an agent targeting BRAF and/or MEK and an immuno-oncology therapy has a high TMB score.

In another aspect, the invention provides a method of stratifying melanoma patients into two groups for selecting a therapy; the method comprising scoring the TMB in a sample from the patient, wherein a low TMB score identifies a patient who may benefit from a targeted therapy comprising an agent targeting BRAF and/or MEK, while high TMB score identifies the patient as one who may benefit from an combination of a targeted therapy comprising an agent targeting BRAF and/or MEK and an immuno-oncology therapy.

In some embodiments of any one of the preceding two aspects, either (a) a low TMB score has been determined and the method further comprises administering the patient an effective amount of a targeted therapy comprising an agent targeting BRAF and/or MEK, or (b) a high TMB score has been determined, and the method further comprises administering the patient an effective amount of a combination of a targeted therapy comprising an agent targeting BRAF and/or MEK and an immuno-oncology therapy.

In another aspect, the invention provides a method of treating a melanoma patient, the method comprising (a) scoring the TMB in a sample from the patient and (b) administering an effective amount of therapy to the patient, wherein the therapy is a targeted therapy comprising an agent targeting BRAF and/or MEK for patients with low TMB score, and the therapy is a combination of a targeted therapy comprising an agent targeting BRAF and/or MEK and an immuno-oncology therapy for patients with a high TMB score.

In another aspect, the invention provides a method of treating a melanoma patient, the method comprising administering to the patient either (a) an effective amount of a targeted therapy comprising an agent targeting BRAF and/or MEK, wherein prior to the administration a low TMB score has been determined from a sample from the patient, or (b) an effective amount of a combination of a targeted therapy comprising an agent targeting BRAF and/or MEK and an immuno-oncology therapy, wherein prior to the administration a high TMB score has been determined from a sample from the patient.

In another aspect, the invention provides a method of treating a patient having melanoma with either (a) a targeted therapy comprising an agent targeting BRAF and/or MEK, wherein the melanoma of said patient is characterized as having a low TMB score, or (b) a combination of a targeted therapy comprising an agent targeting BRAF and/or MEK and an immuno-oncology therapy, wherein the melanoma of said patient is characterized as having a TMB score.

In another aspect, the invention provides the use of TMB as a predictive marker for selecting melanoma patients for treatment with a targeted therapy comprising a BRAF and/or MEK inhibitor in combination with an immuno-oncology treatment, wherein the targeted therapy comprises an agent targeting BRAF and/or MEK and the immuno-oncology treatment is a PD-1 or PD-L1 binding antagonist, comprising scoring the TMB in a sample from said patient.

In another aspect, the invention provides a method of stratifying melanoma patients into two groups, one that may benefit from a targeted therapy comprising an agent targeting BRAF and/or MEK, and second one that may benefit from a combination of a targeted therapy comprising an agent targeting BRAF and/or MEK and an immuno-oncology therapy; the method comprising scoring the i) TMB and ii) immune activation level in a sample from the patient, wherein a patient who may benefit from a targeted therapy comprising an agent targeting BRAF and/or MEK has a low TMB score with a low immune activation score, while a patient who may benefit from a combination of a targeted therapy comprising an agent targeting BRAF and/or MEK and an immuno-oncology therapy has either a low TMB score with a high immune activation score or a high TMB score, irrespective of immune activation score.

In another aspect, the invention provides a method of stratifying melanoma patients into two groups for selecting a therapy; the method comprising scoring i) the TMB and ii) immune activation level in a sample from the patient, wherein a low TMB score with a low immune activation score identifies a patient who may benefit from a targeted therapy comprising an agent targeting BRAF and/or MEK, while either a low TMB score with a high immune activation score or a high TMB score, irrespective of immune activation score identifies the patient as one who may benefit from an combination of a targeted therapy comprising an agent targeting BRAF and/or MEK and an immuno-oncology therapy.

In some embodiments of any one of the preceding two aspects, either (a) a low TMB score with a low immune activation score has been determined and the method further comprises administering the patient an effective amount of a targeted therapy comprising an agent targeting BRAF and/or MEK, or (b) either a low TMB score with a high immune activation score or a high TMB score, irrespective of immune activation score has been determined, and the method further comprises administering the patient an effective amount of a combination of a targeted therapy comprising an agent targeting BRAF and/or MEK and an immuno-oncology therapy.

In another aspect, the invention provides a method of treating a melanoma patient, the method comprising (a) scoring the i) TMB and ii) immune activation level in a sample from the patient and (b) administering an effective amount of therapy to the patient, wherein the therapy is a targeted therapy comprising an agent targeting BRAF and/or MEK for patients with low TMB score with a low immune activation score, and the therapy is a combination of a targeted therapy comprising an agent targeting BRAF and/or MEK and an immuno-oncology therapy for patients with either a low TMB score with a high immune activation score or a high TMB score, irrespective of immune activation score.

In another aspect, the invention provides a method of treating a melanoma patient, the method comprising administering to the patient either (a) an effective amount of a targeted therapy comprising an agent targeting BRAF and/or MEK, wherein prior to the administration a low TMB score with a low immune activation score has been determined from a sample from the patient, or (b) an effective amount of a combination of a targeted therapy comprising an agent targeting BRAF and/or MEK and an immuno-oncology therapy, wherein prior to the administration either a low TMB score with a high immune activation score or a high TMB score, irrespective of immune activation score has been determined from a sample from the patient.

In another aspect, the invention provides a method of treating a patient having melanoma with either (a) a targeted therapy comprising an agent targeting BRAF and/or MEK, wherein the melanoma of said patient is characterized as having a low TMB score with a low immune activation score, or (b) an combination of a targeted therapy comprising an agent targeting BRAF and/or MEK and an immuno-oncology therapy, wherein the melanoma of said patient is characterized as having either a low TMB score with a high immune activation score or a high TMB score, irrespective of immune activation score.

In another aspect, the invention provides the use of TMB and immune activation as a predictive marker for selecting melanoma patients for treatment with a targeted therapy comprising a BRAF and/or MEK inhibitor in combination with an immuno-oncology treatment, wherein the targeted therapy comprises an agent targeting BRAF and/or MEK and the immuno-oncology treatment is a PD-1 or PD-L1 binding antagonist, comprising scoring the i) TMB and ii) immune activation level in a sample from said patient.

### Definitions

### General Definitions

It is to be understood that aspects and embodiments of the invention described herein include “comprising”, “consisting”, and “consisting essentially of” aspects and embodiments.

As used herein, the singular form “a”, “an”, and “the” includes plural references unless indicated otherwise.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”. In some embodiments, “about” indicates a value of up to ±10% of a recited value, e.g. ±1%, ±2%, ±3%, ±4%, ±5%, ±6%, ±7%, ±8%, ±9%, or ±10%.

### TMB in General

Definitions for TMB and its measurements been disclosed in WO 2018/068028, which is hereby incorporated by reference in its entirety.

As used herein, the term “mutational load”, “tumor mutation burden”, or “TMB” may be understood interchangeably and refer to the level (e.g. number) of an alteration (e g. one or more alterations, e.g. one or more somatic alterations) per a pre-selected unit (e.g. per megabase (Mb)) in a pre-determined set of genes (e.g. in the coding regions of the pre-determined set of genes) detected from a tumor (e.g. a tumor tissue sample, e.g. a formalin-fixed and paraffin-embedded (FFPE) tumor sample, an archival tumor sample, a fresh frozen tumor sample, or a blood sample containing tumor cells, tumor RNA, DNA, or proteins). The TMB score can be measured, for example on a whole genome or exome basis, or on the basis of a subset of the genome or exome. In certain embodiments, the TMB score measured on the basis of a subset of the genome or exome can be extrapolated to determine a whole genome or exome mutational load. In some embodiments, a TMB score refers to the level of accumulated somatic mutations within an individual (e.g. an animal, e.g. a human). The TMB score may refer to accumulated somatic mutations in a patient with cancer (e.g. melanoma). In some embodiments, a TMB score refers to the accumulated mutations in the whole genome of an individual. In some embodiments, a TMB score refers to the accumulated mutations within a particular sample (e.g. a tumor sample, e.g. a melanoma sample) collected from a patient.

The term “somatic mutation” or “somatic alteration” refers to a genetic alteration occurring in the somatic tissues (e.g. cells outside the germline). Examples of genetic alterations include, but are not limited to, point mutations (e.g. the exchange of a single nucleotide for another (e.g. silent mutations, missense mutations, and nonsense mutations)), insertions and deletions (e.g. the addition and/or removal of one or more nucleotides (e.g. indels)), amplifications, gene duplications, copy number alterations (CNAs), rearrangements, and splice-site mutations. The presence of particular mutations can be associated with disease states (e.g. cancer, e.g. melanoma).

In certain embodiments, the somatic alteration is a silent mutation (e.g. a synonymous alteration). In other embodiments, the somatic alteration is a non-synonymous single nucleotide variant (SNV). In other embodiments, the somatic alteration is a passenger mutation (e.g. an alteration that has no detectable effect on the fitness of a clone). In certain embodiments, the somatic alteration is a variant of unknown significance (VUS), for example, an alteration, the pathogenicity of which can neither be confirmed nor ruled out. In certain embodiments, the somatic alteration has not been identified as being associated with a cancer phenotype.

In certain embodiments, the somatic alteration is not associated with, or is not known to be associated with, an effect on cell division, growth, or survival. In other embodiments, the somatic alteration is associated with an effect on cell division, growth, or survival.

In certain embodiments, the number of somatic alterations excludes a functional alteration in a sub-genomic interval.

In some embodiments, the functional alteration is an alteration that, compared with a reference sequence (e.g. a wild-type or unmutated sequence) has an effect on cell division, growth, or survival (e.g. promotes cell division, growth, or survival). In certain embodiments, the functional alteration is identified as such by inclusion in a database of functional alterations, e.g. the COSMIC database (see Forbes et al. Nucl. Acids Res. 43 (D1): D805-D811, 2015, which is herein incorporated by reference in its entirety). In other embodiments, the functional alteration is an alteration with known functional status (e.g. occurring as a known somatic alteration in the COSMIC database). In certain embodiments, the functional alteration is an alteration with a likely functional status (e.g. a truncation in a tumor suppressor gene). In certain embodiments, the functional alteration is a driver mutation (e.g. an alteration that gives a selective advantage to a clone in its microenvironment, e.g. by increasing cell survival or reproduction). In other embodiments, the functional alteration is an alteration capable of causing clonal expansions. In certain embodiments, the functional alteration is an alteration capable of causing one, two, three, four, five, or all six of the following: (a) self-sufficiency in a growth signal; (b) decreased, e.g. insensitivity, to an antigrowth signal; (c) decreased apoptosis; (d) increased replicative potential; (e) sustained angiogenesis; or (f) tissue invasion or metastasis.

In certain embodiments, all functional alterations in all genes (e.g. tumor genes) in the pre-determined set of genes are excluded.

In certain embodiments, the number of somatic alterations excludes alterations present below frequency threshold in the sample (e.g. below 5%, below 3%, below 1%).

In certain embodiments, the number of somatic alterations excludes a germline mutation in a sub-genomic interval.

In certain embodiments, the germline alteration is an SNP, a base substitution, an insertion, a deletion, an indel, or a silent mutation (e.g. synonymous mutation).

In certain embodiments, the germline alteration is excluded by use of a method that does not use a comparison with a matched normal sequence. In other embodiments, the germline alteration is excluded by a method comprising the use of an algorithm. In certain embodiments, the germline alteration is identified as such by inclusion in a database of germline alterations, for example, the dbSNP database (see Sherry et al. Nucleic Acids Res. 29(1): 308-311, 2001, which is herein incorporated by reference in its entirety). In other embodiments, the germline alteration is identified as such by inclusion in the ExAC database (see Exome Aggregation Consortium et al. bioRxiv preprint, Oct. 30, 2015, which is herein incorporated by reference in its entirety). In some embodiments, the germline alteration is identified as such by inclusion in the ESP database (Exome Variant Server, NHLBI GO Exome Sequencing Project (ESP), Seattle, Wash.). In some embodiments, the germline alteration is identified by modeling the tumor content of the sample (see Riester et al. Source Code Biol Med. 2016 Dec. 15; 11:13). In some embodiments, the germline alternation is identified from sequencing on samples for individuals who do not have cancer.

Immune Activation in General

The term “immune activation”, “immune activation marker”, “immune activation level”, or “immune activation score” refers to an activation of the intratumoral T-cell adaptive immune response against a tumor (cancer related immunity), which can be characterized and quantified by multiple markers; e.g. by an increase in tumor infiltration lymphocytes (as determined by H/E staining or CD8 gene/protein expression levels), interferon gamma and associated markers, PD-L1 (protein or gene expression), other known checkpoints (e.g. LAG3, TIM3) or a combination of markers in a signature (e.g. IFN gamma, T-cell, inflammation gene expression signature). Response to immuno-therapy occurs primarily in patients with such a preexisting, intratumoral T cell adaptive immune response.

IFN gamma (IFN-γ, IFNy) is a cytokine, which is not only crucial for the host response to viral infections, but also plays a key role in cancer related immunity. IFN-γ is secreted by immune cells in the tumor microenvironment and coordinates the process of innate and adaptive antitumor response (e.g. augments MHC class I expression, contributes to the recruitment of effector cells). At the same time the same IFN-γ signaling processes can induce a feedback inhibition. As part of this feedback loop, IFN-γ signaling enables the PD-1 signaling axis to become activated through direct upregulation of the ligands PD-L1 and PD-L2 in tumor, immune infiltrate, and stromal cells, which ultimately compromises antitumor immunity.

There are various detection methods for immune gene such as CD8 and PD-L1: e.g. IHC, flow cytometry, mRNA expression in samples, e.g. tissue, blood and exosomes. PD-L1 protein testing by immunohistochemistry (IHC) for instance can be done by different antibody clones, e.g. PD-L1 IHC 22C3 PharmDx kit (Dako North America, Carpinteria, Calif., USA), PD-L1 28-8 PharmDx kit (Dako North America) and the PD-L1 SP142 Ventana test (Ventana Medical Systems Inc., Tucson, Ariz., USA). PD-L1 protein levels can be examined e.g. on tumor and immune cells.

“Immune gene expression signatures” combine the expression levels of different T-cell, checkpoints and IFN-γ associated genes. Such signatures may perform favorably compared with single markers (like CD8 and PD-L1) in predicting response to immuno-therapy. Immune gene expression signatures have not been analyzed in melanoma patients treated with adjuvant targeted therapy and the results summarized here show for the first time that immune gene expression signatures together with TMB may help to identify responders to adjuvant targeted therapy in melanoma.

Multiple signatures (e.g. T-cell inflamed, IFNy, T-cell/CD8 gene expression signatures) were described in the literature (e.g. T-cell inflamed in Cristescu et al., “Pan-tumor genomic biomarkers for PD-1 checkpoint blockade-based immunotherapy”, Science 2018). These gene signatures represent a novel method for capturing the complexity of the dynamic immune response to a tumor by distinguishing between tumors with preexisting inflammatory components and noninflamed tumors. Inspection of the gene lists of these signatures suggest that there is a considerable overlap in selected genes, and especially biological features (e.g. including IFN-γ signaling, cytolytic activity, antigen presentation, and T cell trafficking, as well as inhibitory mechanisms that are evident in T cell homeostasis), as all these signatures are highly correlated and identify tumors with an ongoing adaptive Th1 and cytotoxic CD8+ T cell response.

The IFN-γ and CD8/T-cell signatures used in our examples: CCL5, CTSW, FASLG, CD8B, ZNF683, GZMA, XCL2, CD7, KLRC1, CD8A, XCL1, NKG7, KLRK1, GNLY, PRF1, GZMB, GZMH, LAG3, KLRD1 for CD8 T cell signature and IFNG, CXCL9, CXCL10, CXCL11, GBP1 for IFN-γ signature.

It is understood by persons skilled in the art, that these gene expression signatures can be replaced by other T-cell inflamed and IFN-γ signatures (e.g. Cristescu et al., “Pan-tumor genomic biomarkers for PD-1 checkpoint blockade-based immunotherapy”, Science 2018; Ayers et al. IFN-γ-related mRNA profile predicts clinical response to PD-1 blockade, The Journal of Clinical Investigation 2017).

There are several other reported IFNy signatures, one example is the 6-gene signature published by Ayers et al. (IFN-γ-related mRNA profile predicts clinical response to PD-1 blockade, The Journal of Clinical Investigation 2017): IDO1, CXCL10, CXCL9, HLA-DRA, STAT1, IFNG

A well established T cell inflamed signature is composed of 18 inflammatory genes related to antigen presentation, chemokine expression, cytolytic activity, and adaptive immune resistance, including CCL5, CD27, CD274 (PD-L1), CD276 (B7-H3), CD8A, CMKLR1, CXCL9, CXCR6, HLA-DQA1, HLA-DRB1, HLA-E, IDO1, LAG3, NKG7, PDCD1LG2 (PDL2), PSMB10, STAT1, and TIGIT. The T cell inflamed signature can be used to stratify patients into low and high T cell inflamed signature levels (greater than or equal to the cutoff of −0.318=high, less than the −0.318 cutoff=low) (e.g. Cristescu et al., “Pan-tumor genomic biomarkers for PD-1 checkpoint blockade-based immunotherapy”, Science 2018; Ayers et al. IFN-γ-related mRNA profile predicts clinical response to PD-1 blockade, The Journal of Clinical Investigation 2017)

It is understood by persons skilled in the art, that similar cut-offs can be established for other IFN-γ and T cell signatures that are described in this patent description and in the literature. For example, PD-L1 cut-offs of >1%, >5%, >10% (preferably 1%) can be used to define low/high immune activation.

Targeted Therapy

As used herein, “targeted therapy” refers to a cancer therapy using drugs or other substances that block the growth and spread of cancer by interfering with specific molecules (“molecular targets”) that are involved in the growth, progression, relapse, and spread of cancer. Targeted cancer therapies are sometimes called “molecularly targeted drugs”, “molecularly targeted therapies”, “precision medicines”, or similar names.

As used herein, a “targeted therapy comprising agents targeting BRAF and/or MEK” refers to a combination therapy with one or more agents targeting BRAF and one or more agents targeting MEK.

As used herein, an “agent targeting BRAF” refers to an agent that directly or indirectly targets, decreases or inhibits the activity and/or function of BRAF. Exemplary agents targeting BRAF include, but are not limited to, compounds, proteins or antibodies that target BRAF. Preferably, said agent targeting BRAF is a “BRAF inhibitor”.

As used herein, an “agent targeting MEK” refers to an agent that directly or indirectly targets, decreases or inhibits the activity and/or function of MEK. Exemplary agents targeting MEK include, but are not limited to, compounds, proteins or antibodies that target MEK. Preferably, said agent targeting MEK is a “MEK inhibitor”.

Preferably, the BRAF inhibitor is Dabrafenib and the MEK inhibitor is Trametinib. Both molecules as well as their combination are disclosed e.g. in WO 2011/047238 A1, which is hereby incorporated in its entirety by reference.

As used herein, the BRAF inhibitor Dabrafenib N-{3-[5-(2-Amino-4-pyrimidinyl)-2-(1,1-dimethylethyl)-1,3-thiazol-4-yl]-2-fluorophenyl}-2,6-difluorobenzenesulfonamide or pharmaceutically acceptable salt thereof, is represented by a compound formula (II):

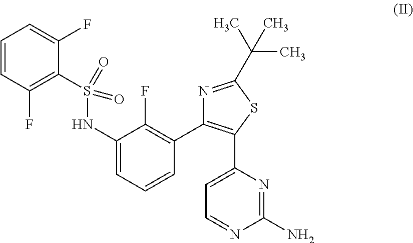 [SMILES: CC(C)(C)c1nc(-c2cccc(NS(=O)(=O)c3c(F)cccc3F)c2F)c(-c2ccnc(N)n2)s1]

or a pharmaceutically acceptable salt thereof. For convenience, the group of possible compounds and salts is collectively referred to as Dabrafenib, meaning that reference to Dabrafenib will refer to any of the compounds or pharmaceutically acceptable salts thereof in the alternative.

Dabrafenib is disclosed and claimed, along with pharmaceutically acceptable salts thereof, as being useful as an inhibitor of BRAF activity, particularly in the treatment of cancer, in PCT patent application PCT/US09/42682. Dabrafenib is embodied by Examples 58a through 58e of the application. The PCT application was published on 12 Nov. 2009 as publication WO2009/137391, and is hereby incorporated by reference.

More particularly, Dabrafenib may be prepared according to the methods from the description of WO 2011/047238 A1 (page 15 to 21), which is hereby incorporated by reference.

As used herein, the MEK inhibitor Trametinib N-{3-[3-cyclopropyl-5-(2-fluoro-4-iodophenylamino)6,8-dimethyl-2,4,7-trioxo-3,4,6,7-tetrahydro-2H-pyrido[4,3-d]pyrimidin-1-yl]phenyl}acetamide, or a pharmaceutically acceptable salt or solvate thereof, is represented by a compound of formula (I):

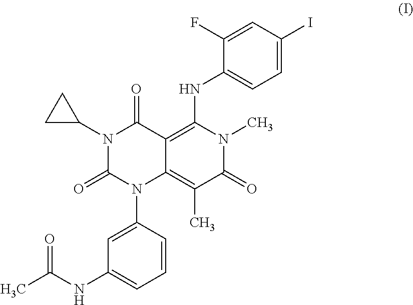 [SMILES: CC(=O)Nc1cccc(-n2c(=O)n(C3CC3)c(=O)c3c(Nc4ccc(I)cc4F)n(C)c(=O)c(C)c32)c1]

or pharmaceutically acceptable salt or solvate thereof. For convenience, the group of possible compounds and salts or solvates is collectively referred to as Trametinib, meaning that reference to Trametinib will refer to any of the compounds or pharmaceutically acceptable salts or solvates thereof in the alternative.

Depending on the naming convention, the compound of formula (I) may also properly be referred to as N-{3-[3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl]phenyl}acetamide.

Trametinib is disclosed and claimed, along with pharmaceutically acceptable salts thereof, and also as solvates thereof, as being useful as an inhibitor of MEK activity, particularly in treatment of cancer, in WO 2005/121 142. Trametinib is the compound of Example 4-1. can be prepared as described in WO 2005/121 142.

Suitably, Trametinib is in the form of a dimethyl sulfoxide solvate. Suitably, Trametinib is in the form of a sodium salt. Suitably, Trametinib is in the form of a solvate selected from: hydrate, acetic acid, ethanol, nitromethane, chlorobenzene, 1-pentancol, isopropyl alcohol, ethylene glycol and 3-methyl-1-butanol. These solvates and salt forms can be prepared by one of skill in the art from the description in WO 2005/121 142.

In another embodiment, the BRAF inhibitor is Vemurafenib and the MEK inhibitor is Cobimetinib. Vemurafenib is disclosed in WO05062795 A2/A3 and WO07013896 A2/A3/WO07002325 A1/WO07002433 A1 and Cobimetinib is disclosed in WO07044515 A1, which are hereby incorporated in their entirety by reference.

As used herein, the MEK inhibitor Vemurafenib, or a pharmaceutically acceptable salt or solvate thereof, is represented by a compound of formula (IV):

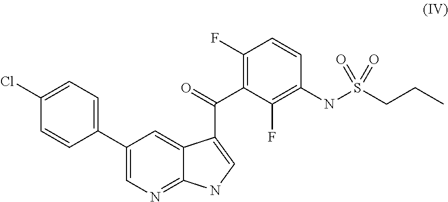 [SMILES: CCCS(=O)(=O)Nc1ccc(F)c(C(=O)c2c[nH]c3ncc(-c4ccc(Cl)cc4)cc23)c1F]

or pharmaceutically acceptable salt or solvate thereof. For convenience, the group of possible compounds and salts or solvates is collectively referred to as Vemurafenib, meaning that reference to Vemurafenib will refer to any of the compounds or pharmaceutically acceptable salts or solvates thereof in the alternative.

Vemurafenib is disclosed and claimed, along with pharmaceutically acceptable salts thereof, and also as solvates thereof, as being useful as an inhibitor of BRAF activity, particularly in the treatment of cancer, in in WO 2007/002325. Vemurafenib may be prepared according to the methods from in WO 2007/002325.

As used herein, the MEK inhibitor Cobimetinib, or a pharmaceutically acceptable salt or solvate thereof, is represented by a compound of formula (III):

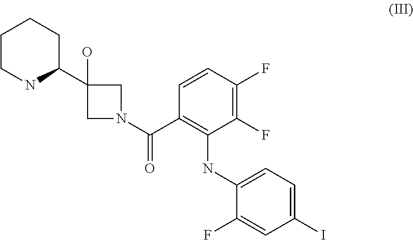 [SMILES: O=C(c1ccc(F)c(F)c1Nc1ccc(I)cc1F)N1CC(O)([C@@H]2CCCCN2)C1]

or pharmaceutically acceptable salt or solvate thereof. For convenience, the group of possible compounds and salts or solvates is collectively referred to as Cobimetinib, meaning that reference to Cobimetinib will refer to any of the compounds or pharmaceutically acceptable salts or solvates thereof in the alternative.

Cobimetinib is disclosed and claimed, along with pharmaceutically acceptable salts thereof, and also as solvates thereof, as being useful as an inhibitor of MEK activity, particularly in treatment of cancer, in WO2007/044515. Cobimetinib is the compound of Example xx. Cobimetinib can be prepared as described in WO2007/044515.

As used herein the term “agent” is understood to mean a substance that produces a desired effect in a tissue, system, animal, mammal, human, or other subject. is also to be understood that an “agent” may be a single compound or a combination or composition of two or more compounds.

Dabrafenib and/or Trametinib, or alternatively Vemurafenib and/or Cobimetinib, may contain one or more chiral atoms, or may otherwise be capable of existing as enantiomers. Accordingly, the compounds of this invention include mixtures of enantiomers as well as purified enantiomers or enantiomerically enriched mixtures. Also, it is understood that all tautomers and mixtures of tautomers are included within the scope of Dabrafenib and Trametinib, or alternatively Vemurafenib and Cobimetinib.

Also, it is understood that Dabrafenib and Trametinib, or alternatively Vemurafenib and Cobimetinib, may be presented, separately or both, as solvates. As used herein, the term “solvate” refers to a complex of variable stoichiometry formed by a solute (in this invention, compounds of formula (I) or (II) or (III) or (IV) or a salt thereof and a solvent. Such solvents for the purpose of the invention may not interfere with the biological activity of the solute. Examples of suitable solvents include, but are not limited to, water, methanol, dimethylsulforide. ethanol and acetic acid. In one embodiment, the solvent used is a pharmaceutically acceptable solvent.

Examples of suitable pharmaceutically acceptable solvents include, without limitation, water, ethanol and acetic acid. In another embodiment, the solvent used is water.

Dabrafenib and Trametinib, or alternatively Vemurafenib and Cobimetinib, may have the ability to crystallize in more than one form, a characteristic, which is known as polymorphism, and it is understood that such polymorphic forms (“polymorphs”) are within the scope of Dabrafenib and Trametinib, or alternatively Vemurafenib and Cobimetinib. Polymorphism generally can occur as a response to changes in temperature or pressure or both and can also result from variations in the crystallization process. Polymorphs can be distinguished by various physical characteristics known in the art such as x-ray diffraction patterns, solubility, and melting point.

Salts encompassed within the term “pharmaceutically acceptable salts” refer to nontoxic salts of the compounds of this invention. Salts of the compounds of the present invention may comprise acid addition salts derived from a nitrogen on a substituent in a compound of the present invention. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucannine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate. Other salts, which are not pharmaceutically acceptable, may be useful in the preparation of compounds of this invention and these form a further aspect of the invention. Salts may be readily prepared by a person skilled in the art.

While it is possible that, for use in therapy, Dabrafenib and Trametinib, or alternatively Vemurafenib and Cobimetinib, may be administered as the raw chemical, it is possible to present the active ingredient as a pharmaceutical composition. Accordingly, the invention further provides pharmaceutical compositions, which include Dabrafenib and/or Trametinib, or alternatively Vemurafenib and/or Cobimetinib, and one or more pharmaceutically acceptable carriers, diluents, or excipients. Dabrafenib and Trametinib, or alternatively Vemurafenib and Cobimetinib are as described above. The carrier(s), diluent(s) or excipient(s) must be acceptable in the sense of being compatible with the other ingredients of the formulation, capable of pharmaceutical formulation, and not deleterious to the recipient thereof. In accordance with another aspect of the invention there is also provided a process for the preparation of a pharmaceutical composition including admixing Dabrafenib and/or Trametinib, or alternatively Vemurafenib and/or Cobimetinib, with one or more pharmaceutically acceptable carriers, diluents or excipients. Such elements of the pharmaceutical compositions utilized may be presented in separate pharmaceutical combinations or formulated together in one pharmaceutical composition. Accordingly, the invention further provides a combination of pharmaceutical compositions one of which includes Dabrafenib, or alternatively Vemurafenib, and one or more pharmaceutically acceptable carriers, diluents, or excipients and a pharmaceutical composition containing Trametinib, or alternatively Cobimetinib, and one or more pharmaceutically acceptable carriers, diluents, or excipients.

Dabrafenib and Trametinib may be employed in combination in accordance with the invention by administration simultaneously in a unitary pharmaceutical composition including both compounds. Likewise, Vemurafenib and Cobimetinib may be employed in combination in accordance with the invention by administration simultaneously in a unitary pharmaceutical composition including both compounds. Alternatively, the combination may be administered separately in separate pharmaceutical compositions, each including either (i) one of the inhibitors, Dabrafenib and Trametinib, in a sequential manner wherein, for example, Dabrafenib and Trametinib is administered first and the other second, or alternatively (ii) one of the inhibitors, Vemurafenib and Cobimetinib, in a sequential manner wherein, for example, Vemurafenib and Cobimetinib is administered first and the other second. Such sequential administration may be close in time (e.g. simultaneously) or remote in time.

Furthermore, it does not matter if the combined compounds are administered in the same dosage form, e.g. one compound may be administered topically and the other compound may be administered orally. Suitably, both compounds are administered orally.

Immuno-Oncology Therapy

As used herein, an “immuno-oncology therapy” is an “immune checkpoint inhibitor” refers to a therapeutic agent that targets at least one immune checkpoint protein to alter the regulation of an immune response, e.g. down-modulating or inhibiting an immune response. Immune checkpoint proteins are known in the art and include, without limitation, cytotoxic T-lymphocyte antigen 4 (CTLA-4), programmed cell death 1 (PD-1), programmed cell death ligand 1 (PD-L1), programmed cell death ligand 2 (PD-L2), V-domain Ig suppressor of T cell activation (VISTA), B7-H2, B7-H3, B7-H4, B7-H6, 2B4, ICOS, HVEM, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, LAG-3, BTLA, IDO, OX40, and A2aR. In some instances, an immune checkpoint protein may be expressed on the surface of an activated T cell. Therapeutic agents that can act as immune checkpoint inhibitors useful in the methods of the present invention, include, but are not limited to, therapeutic agents that target one or more of CTLA-4, PD-1, PD-L1, PD-L2, VISTA, B7-H2, B7-H3, B7-H4, B7-H6, 2B4, ICOS, HVEM, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, LAG-3, BTLA, IDO, OX40, and A2aR. In some instances, an immune checkpoint inhibitor enhances or suppresses the function of one or more targeted immune checkpoint proteins. In some instances, the immune checkpoint inhibitor is a PD-L1 axis binding antagonist as described herein.

In certain embodiments, a combination described herein comprises a PD-1 inhibitor. In some embodiments, the PD-1 inhibitor is chosen from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune). In some embodiments, the PD-1 inhibitor is PDR001. PDR001 is also known as Spartalizumab. Nivolumab (clone 5C4) and other anti-PD-1 antibodies are disclosed in U.S. Pat. No. 8,008,449 and WO 2006/121168, incorporated by reference in their entirety. Pembrolizumab and other anti-PD-1 antibodies are disclosed in Hamid, O. et al. (2013) New England Journal of Medicine 369 (2): 134-44, U.S. Pat. No. 8,354,509, and WO 2009/114335, incorporated by reference in their entirety. Pidilizumab and other anti-PD-1 antibodies are disclosed in Rosenblatt, J. et al. (2011) J Immunotherapy 34(5): 409-18, U.S. Pat. Nos. 7,695,715, 7,332,582, and 8,686,119, incorporated by reference in their entirety. MEDI0680 and other anti-PD-1 antibodies are disclosed in U.S. Pat. No. 9,205,148 and WO 2012/145493, incorporated by reference in their entirety. Further known anti-PD-1 antibodies include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entirety.

In certain embodiments, a combination described herein comprises a PD-1L inhibitor. In some imbodiments the PD-1 L inhibitor is chosen from FAZ053 (Novartis), Atezolizumab (Genentech/Roche), also known as MPDL3280A, RG7446, R05541267, YW243.55.S70, or TECENTRIQ™, Avelumab (Merck Serono and Pfizer), also known as MSB0010718C, Durvalumab (MedImmune/AstraZeneca) also known as MED14736, or BMS-936559 (Bristol-Myers Squibb), also known as MDX-1105 or 12A4. Atezolizumab and other anti-PD-L1 antibodies are disclosed in U.S. Pat. No. 8,217,149, incorporated by reference in its entirety. Avelumab and other anti-PD-L1 antibodies are disclosed in WO 2013/079174, incorporated by reference in its entirety. Durvalumab and other anti-PD-L1 antibodies are disclosed in U.S. Pat. No. 8,779,108, incorporated by reference in its entirety. BMS-936559 and other anti-PD-L1 antibodies are disclosed in U.S. Pat. No. 7,943,743 and WO 2015/081158, incorporated by reference in their entirety.

In one embodiment, the PD-1 inhibitor is an anti-PD-1 antibody molecule. In one embodiment, the PD-1 inhibitor is an anti-PD-1 antibody molecule as described in US 2015/0210769, incorporated by reference in its entirety. In some embodiments, the anti-PD-1 antibody molecule is Spartalizumab (PDR001).

In one embodiment, the anti-PD-1 antibody molecule comprises at least one, two, three, four, five or six complementarity determining regions (CDRs) (or collectively all of the CDRs) from a heavy and light chain variable region comprising an amino acid sequence shown in Table 1 (e.g. from the heavy and light chain variable region sequences of BAP049-Clone-E or BAP049-Clone-B disclosed in Table 1), or encoded by a nucleotide sequence shown in Table 1. In some embodiments, the CDRs are according to the Kabat definition (e.g. as set out in Table 1). In some embodiments, the CDRs are according to the Chothia definition (e.g. as set out in Table 1). In some embodiments, the CDRs are according to the combined CDR definitions of both Kabat and Chothia (e.g. as set out in Table 1). In one embodiment, the combination of Kabat and Chothia CDR of VH CDR1 comprises the amino acid sequence GYTFTTYWMH (SEQ ID NO: 541). In one embodiment, one or more of the CDRs (or collectively all of the CDRs) have one, two, three, four, five, six or more changes, e.g. amino acid substitutions (e.g. conservative amino acid substitutions) or deletions, relative to an amino acid sequence shown in Table 1, or encoded by a nucleotide sequence shown in Table 1.

In one embodiment, the anti-PD-1 antibody molecule comprises a heavy chain variable region (VH) comprising a VHCDR1 amino acid sequence of SEQ ID NO: 501, a VHCDR2 amino acid sequence of SEQ ID NO: 502, and a VHCDR3 amino acid sequence of SEQ ID NO: 503; and a light chain variable region (VL) comprising a VLCDR1 amino acid sequence of SEQ ID NO: 510, a VLCDR2 amino acid sequence of SEQ ID NO: 511, and a VLCDR3 amino acid sequence of SEQ ID NO: 512, each disclosed in Table 1.

In one embodiment, the antibody molecule comprises a VH comprising a VHCDR1 encoded by the nucleotide sequence of SEQ ID NO: 524, a VHCDR2 encoded by the nucleotide sequence of SEQ ID NO: 525, and a VHCDR3 encoded by the nucleotide sequence of SEQ ID NO: 526; and a VL comprising a VLCDR1 encoded by the nucleotide sequence of SEQ ID NO: 529, a VLCDR2 encoded by the nucleotide sequence of SEQ ID NO: 530, and a VLCDR3 encoded by the nucleotide sequence of SEQ ID NO: 531, each disclosed in Table 1.

In one embodiment, the anti-PD-1 antibody molecule comprises a VH comprising the amino acid sequence of SEQ ID NO: 506, or an amino acid sequence at least 85%, 90%, 95%, or 99% identical or higher to SEQ ID NO: 506. In one embodiment, the anti-PD-1 antibody molecule comprises a VL comprising the amino acid sequence of SEQ ID NO: 520, or an amino acid sequence at least 85%, 90%, 95%, or 99% identical or higher to SEQ ID NO: 520. In one embodiment, the anti-PD-1 antibody molecule comprises a VL comprising the amino acid sequence of SEQ ID NO: 516, or an amino acid sequence at least 85%, 90%, 95%, or 99% identical or higher to SEQ ID NO: 516. In one embodiment, the anti-PD-1 antibody molecule comprises a VH comprising the amino acid sequence of SEQ ID NO: 506 and a VL comprising the amino acid sequence of SEQ ID NO: 520. In one embodiment, the anti-PD-1 antibody molecule comprises a VH comprising the amino acid sequence of SEQ ID NO: 506 and a VL comprising the amino acid sequence of SEQ ID NO: 516.

In one embodiment, the antibody molecule comprises a VH encoded by the nucleotide sequence of SEQ ID NO: 507, or a nucleotide sequence at least 85%, 90%, 95%, or 99% identical or higher to SEQ ID NO: 507. In one embodiment, the antibody molecule comprises a VL encoded by the nucleotide sequence of SEQ ID NO: 521 or 517, or a nucleotide sequence at least 85%, 90%, 95%, or 99% identical or higher to SEQ ID NO: 521 or 517. In one embodiment, the antibody molecule comprises a VH encoded by the nucleotide sequence of SEQ ID NO: 507 and a VL encoded by the nucleotide sequence of SEQ ID NO: 521 or 517.

In one embodiment, the anti-PD-1 antibody molecule comprises a heavy chain comprising the amino acid sequence of SEQ ID NO: 508, or an amino acid sequence at least 85%, 90%, 95%, or 99% identical or higher to SEQ ID NO: 508. In one embodiment, the anti-PD-1 antibody molecule comprises a light chain comprising the amino acid sequence of SEQ ID NO: 522, or an amino acid sequence at least 85%, 90%, 95%, or 99% identical or higher to SEQ ID NO: 522. In one embodiment, the anti-PD-1 antibody molecule comprises a light chain comprising the amino acid sequence of SEQ ID NO: 518, or an amino acid sequence at least 85%, 90%, 95%, or 99% identical or higher to SEQ ID NO: 518. In one embodiment, the anti-PD-1 antibody molecule comprises a heavy chain comprising the amino acid sequence of SEQ ID NO: 508 and a light chain comprising the amino acid sequence of SEQ ID NO: 522. In one embodiment, the anti-PD-1 antibody molecule comprises a heavy chain comprising the amino acid sequence of SEQ ID NO: 508 and a light chain comprising the amino acid sequence of SEQ ID NO: 518.

In one embodiment, the antibody molecule comprises a heavy chain encoded by the nucleotide sequence of SEQ ID NO: 509, or a nucleotide sequence at least 85%, 90%, 95%, or 99% identical or higher to SEQ ID NO: 509. In one embodiment, the antibody molecule comprises a light chain encoded by the nucleotide sequence of SEQ ID NO: 523 or 519, or a nucleotide sequence at least 85%, 90%, 95%, or 99% identical or higher to SEQ ID NO: 523 or 519. In one embodiment, the antibody molecule comprises a heavy chain encoded by the nucleotide sequence of SEQ ID NO: 509 and a light chain encoded by the nucleotide sequence of SEQ ID NO: 523 or 519.

The antibody molecules described herein can be made by vectors, host cells, and methods described in US 2015/0210769, incorporated by reference in its entirety.

TABLE 1
Amino acid and nucleotide sequences of exemplary
anti-PD-1 antibody molecules

| | | |
|---|---|---|
| BAP049-Clone-B HC | | |
| SEQ ID NO: 501 | HCDR1 | TYWMH |
| (Kabat) | | |
| SEQ ID NO: 502 | HCDR2 | NIYPGTGGSNFDEKFKN |
| (Kabat) | | |
| SEQ ID NO: 503 | HCDR3 | WTTGTGAY |
| (Kabat) | | |
| SEQ ID NO: 504 | HCDR1 | GYTFTTY |
| (Chothia) | | |
| SEQ ID NO: 505 | HCDR2 | YPGTGG |
| (Chothia) | | |
| SEQ ID NO: 503 | HCDR3 | WTTGTGAY |
| (Chothia) | | |
| SEQ ID NO: 506 | VH | EVQLVQSGAEVKKPGESLRISCKGSGYTFTTYWMHWVRQATGQGLEWMGNIYP |
| | | GTGGSNFDEKFKNRVTITADKSTSTAYMELSSLRSEDTAVYYCTRWTTGTGAY |
| | | WGQGTTVTVSS |
| SEQ ID NO: 507 | DNA | Gaggtgcagctggtgcagtcaggcgccgaagtgaagaagcccggcgagtcact |
| | VH | gagaattagctgtaaaggttcaggctacaccttcactacctactggatgcact |
| | | gggtccgccaggctaccggtcaaggcctcgagtggatgggtaatatctacccc |
| | | ggcaccggcggctctaacttcgacgagaagtttaagaatagagtgactatcac |
| | | cgccgataagtctactagcaccgcctatatggaactgtctagcctgagatcag |
| | | aggacaccgccgtctactactgcactaggtggactaccggcacaggcgcctac |
| | | tggggtcaaggcactaccgtgaccgtgtctagc |
| SEQ ID NO: 508 | Heavy | EVQLVQSGAEVKKPGESLRISCKGSGYTFTTYWMHWVRQATGQGLEWMGNIYP |
| | chain | GTGGSNFDEKFKNRVTITADKSTSTAYMELSSLRSEDTAVYYCTRWTTGTGAY |
| | | WGQGTTVTVSSASTKGPSVFPLAPCSRSTSESTAALGCLVKDYFPEPVTVSWN |
| | | SGALTSGVHTFPAVLQSSGLYSLSSVVTVPSSSLGTKTYTCNVDHKPSNTKVD |
| | | KRVESKYGPPCPPCPAPEFLGGPSVFLFPPKPKDTLMISRTPEVTCVVVDVSQ |
| | | EDPEVQFNWYVDGVEVHNAKTKPREEQFNSTYRVVSVLTVLHQDWLNGKEYKC |
| | | KVSNKGLPSSIEKTISKAKGQPREPQVYTLPPSQEEMTKNQVSLTCLVKGFYP |
| | | SDIAVEWESNGQPENNYKTTPPVLDSDGSFFLYSRLTVDKSRWQEGNVFSCSV |
| | | MHEALHNHYTQKSLSLSLG |
| SEQ ID NO: 509 | DNA | gaggtgcagctggtgcagtcaggcgccgaagtgaagaagcccggcgagtcact |
| | heavy | gagaattagctgtaaaggttcaggctacaccttcactacctactggatgcact |
| | chain | gggtccgccaggctaccggtcaaggcctcgagtggatgggtaatatctacccc |
| | | ggcaccggcggctctaacttcgacgagaagtttaagaatagagtgactatcac |
| | | cgccgataagtctactagcaccgcctatatggaactgtctagcctgagatcag |
| | | aggacaccgccgtctactactgcactaggtggactaccggcacaggcgcctac |
| | | tggggtcaaggcactaccgtgaccgtgtctagcgctagcactaagggcccgtc |
| | | cgtgttccccctggcaccttgtagccggagcactagcgaatccaccgctgccc |
| | | tcggctgcctggtcaaggattacttcccggagcccgtgaccgtgtcctggaac |
| | | agcggagccctgacctccggagtgcacaccttccccgctgtgctgcagagctc |
| | | cgggctgtactcgctgtcgtcggtggtcacggtgccttcatctagcctgggta |
| | | ccaagacctacacttgcaacgtggaccacaagccttccaacactaaggtggac |
| | | aagcgcgtcgaatcgaagtacggcccaccgtgcccgccttgtcccgcgccgga |
| | | gttcctcggcggtccctcggtctttctgttcccaccgaagcccaaggacactt |
| | | tgatgatttcccgcacccctgaagtgacatgcgtggtcgtggacgtgtcacag |
| | | gaagatccggaggtgcagttcaattggtacgtggatggcgtcgaggtgcacaa |
| | | cgccaaaaccaagccgagggaggagcagttcaactccacttaccgcgtcgtgt |
| | | ccgtgctgacggtgctgcatcaggactggctgaacgggaaggagtacaagtgc |
| | | aaagtgtccaacaagggacttcctagctcaatcgaaaagaccatctcgaaagc |
| | | caagggacagccccgggaaccccaagtgtataccctgccaccgagccaggaag |
| | | aaatgactaagaaccaagtctcattgacttgccttgtgaagggcttctaccca |
| | | tcggatatcgccgtggaatgggagtccaacggccagccggaaaacaactacaa |
| | | gaccacccctccggtgctggactcagacggatccttcttcctctactcgcggc |
| | | tgaccgtggataagagcagatggcaggagggaaatgtgttcagctgttctgtg |
| | | atgcatgaagccctgcacaaccactacactcagaagtccctgtccctctccct |
| | | ggga |
| BAP049-Clone-B LC | | |
| SEQ ID NO: 510 | LCDR1 | KSSQSLLDSGNQKNFLT |
| (Kabat) | | |
| SEQ ID NO: 511 | LCDR2 | WASTRES |
| (Kabat) | | |
| SEQ ID NO: 512 | LCDR3 | QNDYSYPYT |
| (Kabat) | | |
| SEQ ID NO: 513 | LCDR1 | SQSLLDSGNQKNF |
| (Chothia) | | |
| SEQ ID NO: 514 | LCDR2 | WAS |
| (Chothia) | | |
| SEQ ID NO: 515 | LCDR3 | DYSYPY |
| (Chothia) | | |
| SEQ ID NO: 516 | VL | EIVLTQSPATLSLSPGERATLSCKSSQSLLDSGNQKNFLTWYQQKPGKAPKLL |
| | | IYWASTRESGVPSRFSGSGSGTDFTFTISSLQPEDIATYYCQNDYSYPYTFGQ |
| | | GTKVEIK |
| SEQ ID NO: 517 | DNA | Gagatcgtcctgactcagtcacccgctaccctgagcctgagccctggcgagcg |
| | VL | ggctacactgagctgtaaatctagtcagtcactgctggatagcggtaatcaga |
| | | agaacttcctgacctggtatcagcagaagcccggtaaagcccctaagctgctg |
| | | atctactgggcctctactagagaatcaggcgtgccctctaggtttagcggtag |
| | | cggtagtggcaccgacttcaccttcactatctctagcctgcagcccgaggata |
| | | tcgctacctactactgtcagaacgactatagctacccctacaccttcggtcaa |
| | | ggcactaaggtcgagattaag |
| SEQ ID NO: 518 | Light | EIVLTQSPATLSLSPGERATLSCKSSQSLLDSGNQKNFLTWYQQKPGKAPKLL |
| | chain | IYWASTRESGVPSRFSGSGSGTDFTFTISSLQPEDIATYYCQNDYSYPYTFGQ |
| | | GTKVEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSPVT |
| | | KSFNRGEC |
| SEQ ID NO: 519 | DNA | Gagatcgtcctgactcagtcacccgctaccctgagcctgagccctggcgagcg |
| | light | ggctacactgagctgtaaatctagtcagtcactgctggatagcggtaatcaga |
| | chain | agaacttcctgacctggtatcagcagaagcccggtaaagcccctaagctgctg |
| | | atctactgggcctctactagagaatcaggcgtgccctctaggtttagcggtag |
| | | cggtagtggcaccgacttcaccttcactatctctagcctgcagcccgaggata |
| | | tcgctacctactactgtcagaacgactatagctacccctacaccttcggtcaa |
| | | ggcactaaggtcgagattaagcgtacggtggccgctcccagcgtgttcatctt |
| | | cccccccagcgacgagcagctgaagagcggcaccgccagcgtggtgtgcctgc |
| | | tgaacaacttctacccccgggaggccaaggtgcagtggaaggtggacaacgcc |
| | | ctgcagagcggcaacagccaggagagcgtcaccgagcaggacagcaaggactc |
| | | cacctacagcctgagcagcaccctgaccctgagcaaggccgactacgagaagc |
| | | ataaggtgtacgcctgcgaggtgacccaccagggcctgtccagccccgtgacc |
| | | aagagcttcaacaggggcgagtgc |
| BAP049-Clone-E HC | | |
| SEQ ID NO: 501 | HCDR1 | TYWMH |
| (Kabat) | | |
| SEQ ID NO: 502 | HCDR2 | NIYPGTGGSNFDEKFKN |
| (Kabat) | | |
| SEQ ID NO: 503 | HCDR3 | WTTGTGAY |
| (Kabat) | | |
| SEQ ID NO: 504 | HCDR1 | GYTFTTY |
| (Chothia) | | |
| SEQ ID NO: 505 | HCDR2 | YPGTGG |
| (Chothia) | | |
| SEQ ID NO: 503 | HCDR3 | WTTGTGAY |
| (Chothia) | | |
| SEQ ID NO: 506 | VH | EVQLVQSGAEVKKPGESLRISCKGSGYTFTTYWMHWVRQATGQGLEWMGNIYP |
| | | GTGGSNFDEKFKNRVTITADKSTSTAYMELSSLRSEDTAVYYCTRWTTGTGAY |
| | | WGQGTTVTVSS |
| SEQ ID NO: 507 | DNA | Gaggtgcagctggtgcagtcaggcgccgaagtgaagaagcccggcgagtcact |
| | VH | gagaattagctgtaaaggttcaggctacaccttcactacctactggatgcact |
| | | gggtccgccaggctaccggtcaaggcctcgagtggatgggtaatatctacccc |
| | | ggcaccggcggctctaacttcgacgagaagtttaagaatagagtgactatcac |
| | | cgccgataagtctactagcaccgcctatatggaactgtctagcctgagatcag |
| | | aggacaccgccgtctactactgcactaggtggactaccggcacaggcgcctac |
| | | tggggtcaaggcactaccgtgaccgtgtctagc |
| SEQ ID NO: 508 | Heavy | EVQLVQSGAEVKKPGESLRISCKGSGYTFTTYWMHWVRQATGQGLEWMGNIYP |
| | chain | GTGGSNFDEKFKNRVTITADKSTSTAYMELSSLRSEDTAVYYCTRWTTGTGAY |
| | | WGQGTTVTVSSASTKGPSVFPLAPCSRSTSESTAALGCLVKDYFPEPVTVSWN |
| | | SGALTSGVHTFPAVLQSSGLYSLSSVVTVPSSSLGTKTYTCNVDHKPSNTKVD |
| | | KRVESKYGPPCPPCPAPEFLGGPSVFLFPPKPKDTLMISRTPEVTCVVVDVSQ |
| | | EDPEVQFNWYVDGVEVHNAKTKPREEQFNSTYRVVSVLTVLHQDWLNGKEYKC |
| | | KVSNKGLPSSIEKTISKAKGQPREPQVYTLPPSQEEMTKNQVSLTCLVKGFYP |
| | | SDIAVEWESNGQPENNYKTTPPVLDSDGSFFLYSRLTVDKSRWQEGNVFSCSV |
| | | MHEALHNHYTQKSLSLSLG |
| SEQ ID NO: 509 | DNA | gaggtgcagctggtgcagtcaggcgccgaagtgaagaagcccggcgagtcact |
| | heavy | gagaattagctgtaaaggttcaggctacaccttcactacctactggatgcact |
| | chain | gggtccgccaggctaccggtcaaggcctcgagtggatgggtaatatctacccc |
| | | ggcaccggcggctctaacttcgacgagaagtttaagaatagagtgactatcac |
| | | cgccgataagtctactagcaccgcctatatggaactgtctagcctgagatcag |
| | | aggacaccgccgtctactactgcactaggtggactaccggcacaggcgcctac |
| | | tggggtcaaggcactaccgtgaccgtgtctagcgctagcactaagggcccgtc |
| | | cgtgttccccctggcaccttgtagccggagcactagcgaatccaccgctgccc |
| | | tcggctgcctggtcaaggattacttcccggagcccgtgaccgtgtcctggaac |
| | | agcggagccctgacctccggagtgcacaccttccccgctgtgctgcagagctc |
| | | cgggctgtactcgctgtcgtcggtggtcacggtgccttcatctagcctgggta |
| | | ccaagacctacacttgcaacgtggaccacaagccttccaacactaaggtggac |
| | | aagcgcgtcgaatcgaagtacggcccaccgtgcccgccttgtcccgcgccgga |
| | | gttcctcggcggtccctcggtctttctgttcccaccgaagcccaaggacactt |
| | | tgatgatttcccgcacccctgaagtgacatgcgtggtcgtggacgtgtcacag |
| | | gaagatccggaggtgcagttcaattggtacgtggatggcgtcgaggtgcacaa |
| | | cgccaaaaccaagccgagggaggagcagttcaactccacttaccgcgtcgtgt |
| | | ccgtgctgacggtgctgcatcaggactggctgaacgggaaggagtacaagtgc |
| | | aaagtgtccaacaagggacttcctagctcaatcgaaaagaccatctcgaaagc |
| | | caagggacagccccgggaaccccaagtgtataccctgccaccgagccaggaag |
| | | aaatgactaagaaccaagtctcattgacttgccttgtgaagggcttctaccca |
| | | tcggatatcgccgtggaatgggagtccaacggccagccggaaaacaactacaa |
| | | gaccacccctccggtgctggactcagacggatccttcttcctctactcgcggc |
| | | tgaccgtggataagagcagatggcaggagggaaatgtgttcagctgttctgtg |
| | | atgcatgaagccctgcacaaccactacactcagaagtccctgtccctctccct |
| | | ggga |
| BAP049-Clone-E LC | | |
| SEQ ID NO: 510 | LCDR1 | KSSQSLLDSGNQKNFLT |
| (Kabat) | | |
| SEQ ID NO: 511 | LCDR2 | WASTRES |
| (Kabat) | | |
| SEQ ID NO: 512 | LCDR3 | QNDYSYPYT |
| (Kabat) | | |
| SEQ ID NO: 513 | LCDR1 | SQSLLDSGNQKNF |
| (Chothia) | | |
| SEQ ID NO: 514 | LCDR2 | WAS |
| (Chothia) | | |
| SEQ ID NO: 515 | LCDR3 | DYSYPY |
| (Chothia) | | |
| SEQ ID NO: 520 | VL | EIVLTQSPATLSLSPGERATLSCKSSQSLLDSGNQKNFLTWYQQKPGQAPRLL |
| | | IYWASTRESGVPSRFSGSGSGTDFTFTISSLEAEDAATYYCQNDYSYPYTFGQ |
| | | GTKVEIK |
| SEQ ID NO: 521 | DNA | Gagatcgtcctgactcagtcacccgctaccctgagcctgagccctggcgagcg |
| | VL | ggctacactgagctgtaaatctagtcagtcactgctggatagcggtaatcaga |
| | | agaacttcctgacctggtatcagcagaagcccggtcaagcccctagactgctg |
| | | atctactgggcctctactagagaatcaggcgtgccctctaggtttagcggtag |
| | | cggtagtggcaccgacttcaccttcactatctctagcctggaagccgaggacg |
| | | ccgctacctactactgtcagaacgactatagctacccctacaccttcggtcaa |
| | | ggcactaaggtcgagattaag |
| SEQ ID NO: 522 | Light | EIVLTQSPATLSLSPGERATLSCKSSQSLLDSGNQKNFLTWYQQKPGQAPRLL |
| | chain | IYWASTRESGVPSRFSGSGSGTDFTFTISSLEAEDAATYYCQNDYSYPYTFGQ |
| | | GTKVEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSPVT |
| | | KSFNRGEC |
| SEQ ID NO: 523 | DNA | Gagatcgtcctgactcagtcacccgctaccctgagcctgagccctggcgagcg |
| | light | ggctacactgagctgtaaatctagtcagtcactgctggatagcggtaatcaga |
| | chain | agaacttcctgacctggtatcagcagaagcccggtcaagcccctagactgctg |
| | | atctactgggcctctactagagaatcaggcgtgccctctaggtttagcggtag |
| | | cggtagtggcaccgacttcaccttcactatctctagcctggaagccgaggacg |
| | | ccgctacctactactgtcagaacgactatagctacccctacaccttcggtcaa |
| | | ggcactaaggtcgagattaagcgtacggtggccgctcccagcgtgttcatctt |
| | | cccccccagcgacgagcagctgaagagcggcaccgccagcgtggtgtgcctgc |
| | | tgaacaacttctacccccgggaggccaaggtgcagtggaaggtggacaacgcc |
| | | ctgcagagcggcaacagccaggagagcgtcaccgagcaggacagcaaggactc |
| | | cacctacagcctgagcagcaccctgaccctgagcaaggccgactacgagaagc |
| | | ataaggtgtacgcctgcgaggtgacccaccagggcctgtccagccccgtgacc |
| | | aagagcttcaacaggggcgagtgc |
| BAP049-Clone-B HC | | |
| SEQ ID NO: 524 | HCDR1 | ACCTACTGGATGCAC |
| (Kabat) | | |
| SEQ ID NO: 525 | HCDR2 | AATATCTACCCCGGCACCGGCGGCTCTAACTTCGACGAGAAGTTTAAGAAT |
| (Kabat) | | |
| SEQ ID NO: 526 | HCDR3 | TGGACTACCGGCACAGGCGCCTAC |
| (Kabat) | | |
| SEQ ID NO: 527 | HCDR1 | GGCTACACCTTCACTACCTAC |
| (Chothia) | | |
| SEQ ID NO: 528 | HCDR2 | TACCCCGGCACCGGCGGC |
| (Chothia) | | |
| SEQ ID NO: 526 | HCDR3 | TGGACTACCGGCACAGGCGCCTAC |
| (Chothia) | | |
| BAP049-Clone-B LC | | |
| SEQ ID NO: 529 | LCDR1 | AAATCTAGTCAGTCACTGCTGGATAGCGGTAATCAGAAGAACTTCCTGACC |
| (Kabat) | | |
| SEQ ID NO: 530 | LCDR2 | TGGGCCTCTACTAGAGAATCA |
| (Kabat) | | |
| SEQ ID NO: 531 | LCDR3 | CAGAACGACTATAGCTACCCCTACACC |
| (Kabat) | | |
| SEQ ID NO: 532 | LCDR1 | AGTCAGTCACTGCTGGATAGCGGTAATCAGAAGAACTTC |
| (Chothia) | | |
| SEQ ID NO: 533 | LCDR2 | TGGGCCTCT |
| (Chothia) | | |
| SEQ ID NO: 534 | LCDR3 | GACTATAGCTACCCCTAC |
| (Chothia) | | |
| BAP049-Clone-E HC | | |
| SEQ ID NO: 524 | HCDR1 | ACCTACTGGATGCAC |
| (Kabat) | | |
| SEQ ID NO: 525 | HCDR2 | AATATCTACCCCGGCACCGGCGGCTCTAACTTCGACGAGAAGTTTAAGAAT |
| (Kabat) | | |
| SEQ ID NO: 526 | HCDR3 | TGGACTACCGGCACAGGCGCCTAC |
| (Kabat) | | |
| SEQ ID NO: 527 | HCDR1 | GGCTACACCTTCACTACCTAC |
| (Chothia) | | |
| SEQ ID NO: 528 | HCDR2 | TACCCCGGCACCGGCGGC |
| (Chothia) | | |
| SEQ ID NO: 526 | HCDR3 | TGGACTACCGGCACAGGCGCCTAC |
| (Chothia) | | |
| BAP049-Clone-E LC | | |
| SEQ ID NO: 529 | LCDR1 | AAATCTAGTCAGTCACTGCTGGATAGCGGTAATCAGAAGAACTTCCTGACC |
| (Kabat) | | |
| SEQ ID NO: 530 | LCDR2 | TGGGCCTCTACTAGAGAATCA |
| (Kabat) | | |
| SEQ ID NO: 531 | LCDR3 | CAGAACGACTATAGCTACCCCTACACC |
| (Kabat) | | |
| SEQ ID NO: 532 | LCDR1 | AGTCAGTCACTGCTGGATAGCGGTAATCAGAAGAACTTC |
| (Chothia) | | |
| SEQ ID NO: 533 | LCDR2 | TGGGCCTCT |
| (Chothia) | | |
| SEQ ID NO: 534 | LCDR3 | GACTATAGCTACCCCTAC |
| (Chothia) | | |

In some embodiments, the PD-1 inhibitor is administered at a dose of about 200 mg to about 500 mg (e.g. about 300 mg to about 400 mg). In some embodiments, the PD-1 inhibitor is administered once every 3 weeks. In some embodiments, the PD-1 inhibitor is administered once every 4 weeks. In other embodiments, the PD-1 inhibitor is administered at a dose of about 200 mg to about 400 mg (e.g. about 300 mg) once every 3 weeks. In yet other embodiments, the PD-1 inhibitor is administered at a dose of about 300 mg to about 500 mg (e.g. about 400 mg) once every 4 weeks.

In one embodiment, the anti-PD-1 antibody molecule is Nivolumab (Bristol-Myers Squibb), also known as MDX-1106, MDX-1106-04, ONO-4538, BMS-936558, or OPDIVO@. Nivolumab (clone 5C4) and other anti-PD-1 antibodies are disclosed in U.S. Pat. No. 8,008,449 and WO 2006/121168, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Nivolumab, e.g. as disclosed in Table 2.

In one embodiment, the anti-PD-1 antibody molecule is Pembrolizumab (Merck & Co), also known as Lambrolizumab, MK-3475, MK03475, SCH-900475, or KEYTRUDA®. Pembrolizumab and other anti-PD-1 antibodies are disclosed in Hamid, O. et al. (2013) New England Journal of Medicine 369 (2): 134-44, U.S. Pat. No. 8,354,509, and WO 2009/114335, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pembrolizumab, e.g. as disclosed in Table 2.

In one embodiment, the anti-PD-1 antibody molecule is Pidilizumab (CureTech), also known as CT-011. Pidilizumab and other anti-PD-1 antibodies are disclosed in Rosenblatt, J. et al. (2011) J Immunotherapy 34(5): 409-18, U.S. Pat. Nos. 7,695,715, 7,332,582, and 8,686,119, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pidilizumab, e.g. as disclosed in Table 2.

In one embodiment, the anti-PD-1 antibody molecule is MEDI0680 (Medimmune), also known as AMP-514. MEDI0680 and other anti-PD-1 antibodies are disclosed in U.S. Pat. No. 9,205,148 and WO 2012/145493, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of MEDI0680.

In one embodiment, the anti-PD-1 antibody molecule is REGN2810 (Regeneron). In one embodiment, the anti-PD-1 antibody molecule comprises one or more of the CDR sequences (or collectively all of the CDR sequences), the heavy chain or light chain variable region sequence, or the heavy chain or light chain sequence of REGN2810.

In one embodiment, the anti-PD-1 antibody molecule is PF-06801591 (Pfizer). In one 40 embodiment, the anti-PD-1 antibody molecule comprises one or more of the CDR sequences (or collectively all of the CDR sequences), the heavy chain or light chain variable region sequence, or the heavy chain or light chain sequence of PF-06801591.

In one embodiment, the anti-PD-1 antibody molecule is BGB-A317 or BGB-108 (Beigene). In one embodiment, the anti-PD-1 antibody molecule comprises one or more of the CDR sequences (or collectively all of the CDR sequences), the heavy chain or light chain variable region sequence, or the heavy chain or light chain sequence of BGB-A317 or BGB-108.

In one embodiment, the anti-PD-1 antibody molecule is INCSHR1210 (Incyte), also known as INCSHR01210 or SHR-1210. In one embodiment, the anti-PD-1 antibody molecule comprises one or more of the CDR sequences (or collectively all of the CDR sequences), the heavy chain or light chain variable region sequence, or the heavy chain or light chain sequence of INCSHR1210.

In one embodiment, the anti-PD-1 antibody molecule is TSR-042 (Tesaro), also known as ANB011. In one embodiment, the anti-PD-1 antibody molecule comprises one or more of the CDR sequences (or collectively all of the CDR sequences), the heavy chain or light chain variable region sequence, or the heavy chain or light chain sequence of TSR-042.

Further known anti-PD-1 antibodies include those described, e.g. in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entirety.

In one embodiment, the anti-PD-1 antibody is an antibody that competes for binding with, and/or binds to the same epitope on PD-1 as, one of the anti-PD-1 antibodies described herein.

In one embodiment, the PD-1 inhibitor is a peptide that inhibits the PD-1 signaling pathway, e.g. as described in U.S. Pat. No. 8,907,053, incorporated by reference in its entirety. In one embodiment, the PD-1 inhibitor is an immunoadhesin (e.g. an immunoadhesin comprising an extracellular or PD-1 binding portion of PD-L1 or PD-L2 fused to a constant region (e.g. an Fc region of an immunoglobulin sequence). In one embodiment, the PD-1 inhibitor is AMP-224 (B7-DCIg (Amplimmune), e.g. disclosed in WO 2010/027827 and WO 2011/066342, incorporated by reference in their entirety).

In one embodiment, the CTLA-4 inhibitor is an antibody that targets CTLA-4. Preferably this antibody is Ipilimumab.

TMB and Immune Activation Measurements

The term “detection” includes any means of detecting, including direct and indirect detection.

The term “biomarker” as used herein refers to an indicator, e.g. predictive, diagnostic, and/or prognostic, which can be detected in a sample, e.g. a particular gene (alteration and expression levels) or protein encoded by said gene, or one or more somatic mutations of said particular gene. The biomarker may serve as an indicator of a particular subtype of disease or disorder (e.g. cancer) characterized by certain molecular, pathological, histological, and/or clinical features (e.g. responsiveness to therapy including targeted therapy comprising an agent targeting BRAF and/or MEK). In some embodiments, a biomarker is a collection of genes or proteins (e.g. single or multiple gene and protein expression levels) or a collective number of mutations/alterations (e.g. somatic mutations) in a collection of genes. Biomarkers include, but are not limited to, polynucleotides, polynucleotide alterations (e.g. polynucleotide copy number alterations), polypeptides, polynucleotide and polypeptide modifications (e.g. post-translational modifications), carbohydrates, and/or glycolipid-based molecular markers.

The “amount” or “level” of the TMB and/or immune activation associated with an increased clinical benefit for an individual is a detectable level in a biological sample. It can be measured by methods known by one skilled in the art and also disclosed herein. The gene or protein expression level (which can be analyzed by methods like IHC, qRT-PCR, Nanostring and other methods known by one skilled in the art) or amount of a somatic mutation can be used to determine the response to the treatment.

The term “level” refers to the amount of a somatic mutation and/or to the amount of immune activation in a biological sample.

“Increased level”, “increased levels”, “elevated level”, “elevated levels”, or “high levels” of somatic mutations and/or immune activation refers to an increased level of somatic mutations and/or immune activation in an individual relative to a control, such as an individual or individuals who are not suffering from the disease or disorder (e.g. cancer) or an internal control (e.g. a reference gene). In some embodiments, increased levels of somatic mutations are present throughout the whole genome of an individual and increased gene or protein expression levels of immune activation markers are detectable. In other embodiments, increased levels of somatic mutations and/or immune activation are present within a sample (e.g. tissue or blood sample) collected from an individual. In some embodiments, the individual has cancer (e.g. melanoma).

“Decreased level”, “decreased levels”, “reduced level”, “reduced levels”, or “low levels” of a somatic mutation and/or immune activation refers to a decreased level of a somatic mutation and/or immune activation in an individual relative to a control, such as an individual or individuals who are not suffering from the disease or disorder (e.g. cancer) or an internal control (e.g. a reference level). In some embodiments, decreased levels of somatic mutations are present throughout the whole genome of an individual. In other embodiments, decreased levels of somatic mutations and/or immune activation are present within a sample (e.g. tissue or blood sample) collected from an individual. In some embodiments, the individual has cancer (e.g. melanoma).

As used herein, a “low TMB score” refers to a TMB score that is at or below a reference TMB score whereas a “high TMB score” refers to a TMB score that is above a reference TMB score.

As used herein, a “low immune activation score” refers to a immune activation score that is at or below a reference immune activation score whereas a “high immune activation score” refers to a immune activation score that is above a reference immune activation score.

As used herein, the term “reference TMB score” refers to a TMB score against which another TMB score is compared, e.g. to make a diagnostic, predictive, prognostic, and/or therapeutic determination. For example, the reference TMB score may be a TMB score in a reference sample, a reference population, and/or a pre-determined value. In some instances, the individual's responsiveness to treatment with a targeted therapy, is significantly improved relative to the individual's responsiveness to treatment with the non-targeted therapy at or below the cutoff value. In some instances, the individual's responsiveness to treatment with the non-targeted therapy is significantly improved relative to the individual's responsiveness to treatment with the targeted therapy, above the cutoff value.

It will be appreciated by one skilled in the art that the numerical value for the reference TMB score may vary depending on the type of cancer (e.g. a lung cancer (e.g. a non-small cell lung cancer (NSCLC) or a small cell lung cancer), a kidney cancer (e.g. a kidney urothelial carcinoma or a renal cell carcinoma (RCC)), a bladder cancer (e.g. a bladder urothelial (transitional cell) carcinoma (e.g. locally advanced or metastatic urothelial carcinoma, including first-line (1L) or second-line or higher (2L+) locally advanced or metastatic urothelial carcinoma)), a breast cancer (e.g. human epidermal growth factor receptor-2 (HER2)+breast cancer or hormone receptor-positive (HR+) breast cancer), a colorectal cancer (e.g. a colon adenocarcinoma), an ovarian cancer, a pancreatic cancer, a gastric carcinoma, an esophageal cancer, a mesothelioma, a melanoma (e.g. a skin melanoma), a skin cancer (e.g. squamous cell carcinoma of the skin) a head and neck cancer (e.g. a head and neck squamous cell carcinoma (HNSCC)), a thyroid cancer, a sarcoma (e.g. a soft-tissue sarcoma, a fibrosarcoma, a myxosarcoma, a liposarcoma, an osteogenic sarcoma, an osteosarcoma, a chondrosarcoma, an angiosarcoma, an endotheliosarcoma, a lymphangiosarcoma, a lymphangioendotheliosarcoma, a leiomyosarcoma, or a rhabdomyosarcoma), a prostate cancer, a glioblastoma, a cervical cancer, a thymic carcinoma, a leukemia (e.g. an acute lymphocytic leukemia (ALL), an acute myelocytic leukemia (AML), a chronic myelocytic leukemia (CML), a chronic eosinophilic leukemia, or a chronic lymphocytic leukemia (CLL)), a lymphoma (e.g. a Hodgkin lymphoma or a non-Hodgkin lymphoma (NHL)), a myeloma (e.g. a multiple myeloma (MM)), a mycosis fungoides, a merkel cell cancer, a hematologic malignancy, a cancer of hematological tissues, a B cell cancer, a bronchus cancer, a stomach cancer, a brain or central nervous system cancer, a peripheral nervous system cancer, a uterine or endometrial cancer, a cancer of the oral cavity or pharynx, a liver cancer, a testicular cancer, a biliary tract cancer, a small bowel or appendix cancer, a salivary gland cancer, an adrenal gland cancer, an adenocarcinoma, an inflammatory myofibroblastic tumor, a gastrointestinal stromal tumor (GIST), a colon cancer, a myelodysplastic syndrome (MDS), a myeloproliferative disorder (MPD), a polycythemia Vera, a chordoma, a synovioma, an Ewing's tumor, a squamous cell carcinoma, a basal cell carcinoma, an adenocarcinoma, a sweat gland carcinoma, a sebaceous gland carcinoma, a papillary carcinoma, a papillary adenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilms' tumor, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a meningioma, a neuroblastoma, a retinoblastoma, a follicular lymphoma, a diffuse large B-cell lymphoma, a mantle cell lymphoma, a hepatocellular carcinoma, a thyroid cancer, a small cell 50 cancer, an essential thrombocythemia, an agnogenic myeloid metaplasia, a hypereosinophilic syndrome, a systemic mastocytosis, a familiar hypereosinophilia, a neuroendocrine cancer, or a carcinoid tumor, the methodology used to measure a TMB score, and/or the statistical methods used to generate a TMB score.

The term “equivalent TMB value” refers to a numerical value that corresponds to a TMB score that can be calculated by dividing the count of somatic variants (e.g. somatic mutations) by the number of bases sequenced (e g. about 1.5 Mb as assessed by a targeted panel). It is to be understood that, in general, the TMB score is linearly related to the size of the genomic region sequenced. Such equivalent TMB values indicate an equivalent degree of tumor mutation burden as compared to a TMB score and can be used interchangeably in the methods described herein, for example, to predict response of a cancer patient to a targeted therapy (e.g. targeted therapy comprising an agent targeting BRAF and/or MEK, e.g. Dabrafenib and Trametinib or e.g. Vemurafenib and Cobimetinib). As an example, in some embodiments, an equivalent TMB value is a normalized TMB value that can be calculated by dividing the count of somatic variants (e.g. somatic mutations) by the number of bases sequenced. For example, an equivalent TMB value can be represented as the number of somatic mutations counted over a defined number of sequenced bases (e.g. about 1.5 Mb as assessed by a targeted panel). It is to be understood that TMB scores as described herein (e.g. TMB scores represented as the number of somatic mutations counted over a defined number of sequenced bases (e.g. 1.5 Mb for the targeted panel described herein) encompass equivalent TMB values obtained using different methodologies (e.g. whole-exome sequencing or whole-genome sequencing). As an example, for a whole-exome panel, the target region may be approximately 50 Mb, and a sample with about 500 somatic mutations detected is an equivalent TMB value to a TMB score of about 10 mutations/Mb.

As used herein, the term “reference immune activation score” refers to a immune activation score against which another immune activation score is compared, e g. to make a diagnostic, predictive, prognostic, and/or therapeutic determination. For example, the reference immune activation score may be an immune activation score in a reference sample, a reference population, and/or a pre-determined value.

The terms “level of expression” or “expression level” in general are used interchangeably and generally refer to the amount of a biomarker in a biological sample. “Expression” generally refers to the process by which information (e.g. gene-encoded and/or epigenetic information) is converted into the structures present and operating in the cell. Therefore, as used herein, “expression” may refer to transcription into a polynucleotide, translation into a polypeptide, or even polynucleotide and/or polypeptide modifications (e.g. posttranslational modification of a polypeptide). Fragments of the transcribed polynucleotide, the translated polypeptide, or polynucleotide and/or polypeptide modifications (e.g. posttranslational modification of a polypeptide) shall also be regarded as expressed whether they originate from a transcript generated by alternative splicing or a degraded transcript, or from a post-translational processing of the polypeptide, e.g. by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

“Increased expression”, “increased expression level”, “increased levels”, “elevated expression”, “elevated expression levels”, or “elevated levels” refers to an increased expression or increased levels of a biomarker in an individual relative to a control, such as an individual or individuals who are not suffering from the disease or disorder (e.g. cancer) or an internal control (e.g. a housekeeping biomarker).

“Decreased expression”, “decreased expression level”, “decreased levels”, “reduced expression”, “reduced expression levels”, or “reduced levels” refers to a decreased expression or decreased levels of a biomarker in an individual relative to a control, such as an individual or individuals who are not suffering from the disease or disorder (e.g. cancer) or an internal control (e.g. a housekeeping biomarker).

“Amplification,” as used herein generally refers to the process of producing multiple copies of a desired sequence. “Multiple copies” mean at least two copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The technique of “polymerase chain reaction” or “PCR” as used herein generally refers to a procedure wherein minute amounts of a specific piece of nucleic acid, RNA and/or DNA, are amplified as described, for example, in U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 51:263 (1987) and Erlich, ed., PCR Technology, (Stockton Press, N Y, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

The term “multiplex-PCR” refers to a single PCR reaction carried out on nucleic acids obtained from a single source (e.g. an individual) using more than one primer set for the purpose of amplifying two or more DNA sequences in a single reaction.

“Quantitative real-time polymerase chain reaction” or “qRT-PCR” refers to a form of PCR wherein the amount of PCR product is measured at each step in a PCR reaction. This technique has been described in various publications including, for example, Cronin et al., Am. J. Pathol. 164(1):35-42 (2004) and Ma et al., Cancer Cell 5:607-616 (2004).

The term “microarray” refers to an ordered arrangement of hybridizable array elements, preferably polynucleotide probes, on a substrate.

The term “diagnosis” is used herein to refer to the identification or classification of a molecular or pathological state, disease or condition (e.g. cancer). For example, “diagnosis” may refer to identification of a particular type of cancer. “Diagnosis” may also refer to the classification of a particular subtype of cancer, for instance, by histopathological criteria, or by molecular features (e.g. a subtype characterized by expression of one or a combination of biomarkers (e.g. particular genes or proteins encoded by said genes)).

The term “sample” or “biological sample”, as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof. In one embodiment, “sample” means “tissue sample” or “cell sample”. In another embodiment, “sample” means “blood sample”.

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. For instance, a “tumor sample” is a tissue sample obtained from a tumor or other cancerous tissue. The tissue sample may contain a mixed population of cell types (e.g. tumor cells and non-tumor cells, cancerous cells and non-cancerous cells). The tissue sample may contain compounds which are not naturally intermixed with the tissue in nature such as preservatives, anticoagulants, buffers, fixatives, nutrients, antibiotics, or the like. In some instances, the tissue sample or tumor tissue sample is not a blood sample or sample or a blood constituent, such as plasma. In a preferred embodiment, the tissue sample or cell sample is a tumor sample.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

A “reference sample”, “reference tissue”, “reference cell”, “control sample”, “control tissue”, or “control cell”, as used herein, refers to a sample, tissue, cell, standard, or level that is used for comparison purposes. In one embodiment, a reference sample, reference tissue, reference cell, control sample, control tissue, or control cell is obtained from a healthy and/or non-diseased part of the body (e.g. tissue or cells) of the same subject or individual. For example, the reference sample, reference tissue, reference cell, control sample, control tissue, or control cell may be healthy and/or non-diseased tissue or cells adjacent to the diseased tissue or cells (e.g. tissue or cells adjacent to a tumor). In another embodiment, a reference sample is obtained from an untreated tissue and/or cell of the body of the same subject or individual. In yet another embodiment, a reference sample, reference tissue, reference cell, control sample, control tissue, or control cell is obtained from a healthy and/or non-diseased part of the body (e.g. tissues or cells) of an individual who is not the same subject or individual. In even another embodiment, a reference sample, reference tissue, reference cell, control sample, control tissue, or control cell is obtained from an untreated tissue and/or cell of the body of an individual who is not the same subject or individual.

By “correlate” or “correlating” is meant comparing, in any way, the performance and/or results of a first analysis or protocol with the performance and/or results of a second analysis or protocol. For example, one may use the results of a first analysis or protocol in carrying out a second protocol and/or one may use the results of a first analysis or protocol to determine whether a second analysis or protocol should be performed. With respect to the embodiment of polypeptide analysis or protocol, one may use the results of the polypeptide expression analysis or protocol to determine whether a specific therapeutic regimen should be performed. With respect to the embodiment of polynucleotide analysis or protocol, one may use the results of the polynucleotide expression analysis or protocol to determine whether a specific therapeutic regimen should be performed.

Response to Treatment

“Individual response” or “response” can be assessed using any endpoint indicating a benefit to the individual, including, without limitation, (1) inhibition, to some extent, of disease progression (e.g. cancer progression), including slowing down or complete arrest; (2) a reduction in tumor size; (3) inhibition (i.e. reduction, slowing down, or complete stopping) of cancer cell infiltration into adjacent peripheral organs and/or tissues; (4) inhibition (i.e. reduction, slowing down, or complete stopping) of metastasis; (5) relief, to some extent, of one or more symptoms associated with the disease or disorder (e.g. cancer); (6) increase or extension in the length of survival, including overall survival, progression free survival, and relapse-free survival; and/or (7) decreased mortality at a given point of time following treatment.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and/or progression-free survival and/or regression-free survival); resulting in an objective response (including a complete response or a partial response); or improving signs or symptoms of cancer. In one embodiment, the level of somatic mutation in tumor cells (e.g. tumor mutation burden (TMB)), for example, as determined using methods disclosed herein, is used to identify a patient who is predicted to have an increased likelihood of being responsive or to have a higher magnitude of a response to treatment with a medicament (e.g. targeted therapy, e.g. targeted therapy comprising an agent targeting BRAF and/or MEK), relative to a patient who does not have the same level of somatic mutations. In one embodiment, a decreased level of somatic mutations in tumor cells, for example as determined using methods disclosed herein is used to identify the patient who is predicted to have an increased likelihood of being responsive to treatment with a medicament (e.g. targeted therapy comprising an agent targeting BRAF and/or MEK), relative to a patient who does not have a decreased level of somatic mutations. In another embodiment, the biomarker (e.g. for immune activation, for example, as determined using IHC or gene expression levels) is used to identify the patient who is predicted to have an increased likelihood of being responsive or to have a higher magnitude of a response to treatment with a medicament (e.g. targeted therapy comprising an agent targeting BRAF and/or MEK), relative to a patient who does not express the biomarker. In one embodiment, the biomarker (e.g. for immune activation, for example, as determined using IHC or gene expression levels) is used to identify the patient who is predicted to have an increase likelihood of being responsive to treatment with a medicament (e.g. targeted therapy comprising an agent targeting BRAF and/or MEK), relative to a patient who does not express the biomarker at the same level. In one embodiment, the presence of the biomarker is used to identify a patient who is more likely to respond or to have a higher magnitude of a response to treatment with a medicament, relative to a patient that does not have the presence of the biomarker. In another embodiment, the presence of the biomarker is used to determine that a patient will have an increased likelihood of benefit from treatment with a medicament, relative to a patient that does not have the presence of the biomarker.

An “objective response” refers to a measurable response, including complete response (CR) or partial response (PR). In some embodiments, the “objective response rate (ORR)” refers to the sum of complete response (CR) rate and partial response (PR) rate.

By “complete response” or “CR” is intended the disappearance of all signs of cancer (e.g. disappearance of all target lesions) in response to treatment. This does not always mean the cancer has been cured.

“Sustained response” refers to the sustained effect on reducing tumor growth after cessation of a treatment. For example, the tumor size may be the same size or smaller as compared to the size at the beginning of the medicament administration phase. In some embodiments, the sustained response has a duration at least the same as the treatment duration, at least 1.5×, 2.0×, 2.5×, or 3.0× length of the treatment duration, or longer.

As used herein, “reducing or inhibiting cancer relapse” means to reduce or inhibit tumor or cancer relapse or tumor or cancer progression. As disclosed herein, cancer relapse and/or cancer progression include, without limitation, cancer metastasis.

The term “survival” refers to the patient remaining alive, and includes overall survival as well as progression-free survival and relapse-free survival.

As used herein, “relapse-free survival” or “RFS” refers to the length of time without any disease recurrence after a complete surgical resection of the tumor. during and after treatment during which no signs or symptoms of the disease that was treated (e.g. cancer) appear.

Relapse-free survival may include the amount of time patients have experienced a complete response or a partial response, as well as the amount of time patients have experienced stable disease.

As used herein, “overall survival” or “OS” refers to the percentage of individuals in a group who are likely to be alive after a particular duration of time.

By “extending survival” is meant increasing overall or progression-free and relapse-free survival in a treated patient relative to an untreated patient (i.e. relative to a patient not treated with the medicament), or relative to a patient who does not have somatic mutations at the designated level, and/or relative to a patient treated with an anti-tumor agent.

The term “substantially the same”, as used herein, denotes a sufficiently high degree of similarity between two numeric values, such that one of skill in the art would consider the difference between the two values to be of little or no biological and/or statistical significance within the context of the biological characteristic measured by said values (e.g. Kd values or mutation levels). The difference between said two values is, for example, less than about 50%, less than about 40%, less than about 30%, less than about 20%, and/or less than about 10%, as a function of the reference/comparator value.

The phrase “substantially different”, as used herein, denotes a sufficiently high degree of difference between two numeric values such that one of skill in the art would consider the difference between the two values to be of statistical significance within the context of the biological characteristic measured by said values (e.g. Kd values or mutation levels). The difference between said two values is, for example, greater than about 10%, greater than about 20%, greater than about 30%, greater than about 40%, and/or greater than about 50%, as a function of the value for the reference/comparator molecule.

A “therapeutically effective amount” refers to an amount of a therapeutic agent to treat or prevent a disease or disorder in a mammal. In the case of cancers, the therapeutically effective amount of the therapeutic agent may reduce the number of cancer cells; reduce the primary tumor size; inhibit (i.e. slow to some extent and preferably stop) cancer cell infiltration into peripheral organs; inhibit (i.e. slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), time to relapse, response rates (e.g. CR and PR), duration of response, and/or quality of life.

A “disorder” is any condition that would benefit from treatment including, but not limited to, chronic and acute disorders or diseases including those pathological conditions which predispose the mammal to the disorder in question.

The terms “cancer” and “cancerous” refer to or describe the physiological condition in mammals that is typically characterized by unregulated cell growth. Included in this definition are benign and malignant cancers. By “early stage cancer” or “early stage tumor” is meant a cancer that is not invasive or metastatic or is classified as a stage I or II cancer. Examples of cancer include, but are not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. Examples of a cancer also include, but are not limited to, a lung cancer (e.g. a non-small cell lung cancer (NSCLC)), a kidney cancer (e.g. a kidney urothelial carcinoma or RCC), a bladder cancer (e.g. a bladder urothelial (transitional cell) carcinoma (e.g. locally advanced or metastatic urothelial cancer, including 1L or 2L+ locally advanced or metastatic urothelial carcinoma), a breast cancer, a colorectal cancer (e.g. a colon adenocarcinoma), an ovarian cancer, a pancreatic cancer, a gastric carcinoma, an esophageal cancer, a mesothelioma, a melanoma (e.g. a skin melanoma), a head and neck cancer (e.g. a head and neck squamous cell carcinoma (HNSCC)), a thyroid cancer, a sarcoma (e.g. a soft-tissue sarcoma, a fibrosarcoma, a myxosarcoma, a liposarcoma, an osteogenic sarcoma, an osteosarcoma, a chondrosarcoma, an angiosarcoma, an endotheliosarcoma, a lymphangiosarcoma, a lymphangioendotheliosarcoma, a leiomyosarcoma, or a rhabdomyosarcoma), a prostate cancer, a glioblastoma, a cervical cancer, a thymic carcinoma, a leukemia (e.g. an acute lymphocytic leukemia (ALL), an acute myelocytic leukemia (AML), a chronic myelocytic leukemia (CML), a chronic eosinophilic leukemia, or a chronic lymphocytic leukemia (CLL)), a lymphoma (e.g. a Hodgkin lymphoma or a non-Hodgkin lymphoma (NHL)), a myeloma (e.g. a multiple myeloma (MM)), a mycosis fungoides, a Merkel cell cancer, a hematologic malignancy, a cancer of hematological tissues, a B cell cancer, a bronchus cancer, a stomach cancer, a brain or central nervous system cancer, a peripheral nervous system cancer, a uterine or endometrial cancer, a cancer of the oral cavity or pharynx, a liver cancer, a testicular cancer, a biliary tract cancer, a small bowel or appendix cancer, a salivary gland cancer, an adrenal gland cancer, an adenocarcinoma, an inflammatory myofibroblastic tumor, a gastrointestinal stromal tumor (GIST), a colon cancer, a myelodysplastic syndrome (MDS), a myeloproliferative disorder (MPD), a polycythemia Vera, a chordoma, a synovioma, an Ewing's tumor, a squamous cell carcinoma, a basal cell carcinoma, an adenocarcinoma, a sweat gland carcinoma, a sebaceous gland carcinoma, a papillary carcinoma, a papillary adenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilms' tumor, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a meningioma, a neuroblastoma, a retinoblastoma, a follicular lymphoma, a diffuse large B-cell lymphoma, a mantle cell lymphoma, a hepatocellular carcinoma, a thyroid cancer, a small cell cancer, an essential thrombocythemia, an agnogenic myeloid metaplasia, a hypereosinophilic syndrome, a systemic mastocytosis, a familiar hypereosinophilia, a neuroendocrine cancer, or a carcinoid tumor. More particular examples of such cancers include early stage I-Ill resectable and unresectable (Stage IIIC) or metastatic (Stage IV) melanoma, lung cancer, including NSCLC, squamous cell cancer (e.g. epithelial squamous cell cancer), lung cancer including small-cell lung cancer (SCLC), and adenocarcinoma of the lung and squamous carcinoma of the lung. In particular examples, the lung cancer is NSCLC, for example a locally advanced or metastatic NSCLC (e.g. stage IIIB NSCLC, stage IV NSCLC, or recurrent NSCLC). In some 50 embodiments, the lung cancer (e.g. NSCLC) is unresectable/inoperable lung cancer (e.g. unresectable NSCLC). Other examples include cancer of the peritoneum, hepatocellular cancer, bladder cancer (e.g. urothelial bladder cancer (e.g. transitional cell or urothelial carcinoma, non-muscle invasive bladder cancer, muscle-invasive bladder cancer, and metastatic bladder cancer) and non-urothelial bladder cancer), gastric or stomach cancer including gastrointestinal cancer, pancreatic cancer, glioblastoma, cervical cancer, ovarian cancer, liver cancer, hepatoma, breast cancer (including metastatic breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, Merkel cell cancer, mycosis fungoides, testicular cancer, esophageal cancer, tumors of the biliary tract, as well as head and neck cancer and hematological malignancies. In some embodiments, the cancer is triple-negative metastatic breast cancer, including any histologically confirmed triple-negative (ER-, PR-, HER2-) adenocarcinoma of the breast.

The term “tumor,” as used herein, refers to all neoplastic cell growth and proliferation, whether malignant or benign, and all pre-cancerous and cancerous cells and tissues. The terms “cancer,” “cancerous,” and “tumor” are not mutually exclusive as referred to herein.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology.

Desirable effects of treatment include, but are not limited to, preventing occurrence or recurrence/relapse of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies (e.g. anti-PD-L1 antibodies and/or anti-PD-1 antibodies) are used to delay development of a disease or to slow the progression of a disease.

The term “cancer therapy” refers to a therapy useful in treating cancer. Examples of anti-cancer therapeutic agents include, but are limited to, cytotoxic agents, chemotherapeutic agents, growth inhibitory agents, agents used in radiation therapy, anti-angiogenesis agents, apoptotic agents, anti-tubulin agents, and other agents to treat cancer, for example, anti-CD20 antibodies, platelet derived growth factor inhibitors (e.g. GLEEVEC™ (imatinib mesylate)), a COX-2 inhibitor (e.g. celecoxib), interferons, cytokines, antagonists (e.g. neutralizing antibodies) that bind to one or more of the following targets PDGFR-β, BlyS, APRIL, BCMA receptor(s), TRAIL/Apo2, other bioactive and organic chemical agents, and the like. Combinations thereof are also included in the invention.

As used herein, a patient who “may benefit” from a certain therapy is one who responds with a higher likelihood or a higher magnitude to that therapy.

As used herein, the terms “individual”, “patient”, and “subject” are used interchangeably and refer to any single animal, more preferably a mammal (including such non-human animals as, for example, dogs, cats, horses, rabbits, zoo animals, cows, pigs, sheep, and non-human primates) for which treatment is desired. In particular embodiments, the individual or patient herein is a human.

As used herein, “administering” and “administration” means a method of giving a dosage of a compound (e.g. an antagonist) or a pharmaceutical composition (e.g. a pharmaceutical composition including an antagonist) to a subject (e.g. a patient). Administering can be by any suitable means, including parenteral, intrapulmonary, and intranasal, and, if desired for local treatment, intralesional administration. Parenteral infusions include, for example, intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

The term “concurrently” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

By “reduce or inhibit” is meant the ability to cause an overall decrease of 20%, 30%, 40%, 50%, 60%, 70%, 75%, 80%, 85%, 90%, 95%, or greater. Reduce or inhibit can refer, for example, to the symptoms of the disorder being treated, the presence or size of metastases, or the size of the primary tumor.

The phrase “based on” when used herein means that the information about one or more biomarkers is used to inform a treatment decision, information provided on a package insert, or marketing/promotional guidance, etc.

### EXAMPLES

### Example 1

Updated relapse-free survival (RFS) and biomarker analysis in the COMBI-AD trial of adjuvant dabrafenib+trametinib (D+T) in patients (pts) with resected BRAF V600-mutant stage Ill melanoma

Background: Primary analysis of COMBI-AD (NCT01682083) showed significant improvement in RFS with adjuvant D+T vs placebo (Pbo; HR, 0.47; P<0.001). We present an RFS analysis with extended follow-up (FU), a cure-rate model, and biomarker analysis.

Methods: COMBI-AD is a randomized, phase 3 trial evaluating 12 mo of adjuvant D+T vs Pbo in pts with resected BRAF V600-mutant stage Ill melanoma. A Weibull mixture cure-rate model was used to estimate the proportion of pts who will not relapse. Mutational landscape and gene expression signatures (GES) were examined in baseline tissue samples by sequencing 570 genes and gene expression profiling (GEP) with a NanoString® panel.

Results: Median FU was 44 mo (D+T) and 42 mo (Pbo); 177/438 pts (41%) in the D+T arm and 254/432 pts (59%) in the Pbo arm had relapsed/died. Median RFS was not reached (NR; 95% Cl, 46.9 mo-NR) with D+T vs 16.6 mo (95% Cl, 12.7-22.1 mo) with Pbo (HR, 0.49 [95% Cl, 0.40-0.59]). Estimated cure rate was 54% (95% Cl, 49%-59%) with D+T vs 37% (95% Cl, 32%-42%) with Pbo. DNA sequencing, GEP, and paired DNA+RNA results were available for 368, 507, and 301 pts, respectively. MAPK pathway gene alterations did not correlate with outcome. Immune GES (eg, interferon [IFN]-γ signature) were strongly prognostic in both arms. High tumor mutational burden (TMB) added positive prognostic value to IFN-γ signature in the Pbo arm (high IFN-γ and high TMB associated with longer RFS), whereas in the D+T arm, IFN-γ gene signature identified pts with longer RFS independently of TMB status. Exploratory analysis of RFS in D+T vs Pbo arms in all TMB/IFN-γ subgroups suggested that low TMB or high TMB/high IFN-γ may be associated with greater RFS benefit than high TMB/low IFN-γ.

Conclusions: The updated RFS and cure-rate model confirm the ongoing benefit of adjuvant D+T. MAPK gene alterations previously associated with targeted therapy resistance were not associated with outcome in the adjuvant setting. TMB and immune GES identified pts at higher risk of relapse in the Pbo arm. The predictive value of these markers with targeted therapy or checkpoint inhibitors warrants further validation in a prospective study.

### Example 2

Genomic landscape, tumor mutation burden (TMB), immune gene expression signatures and clinical outcome after adjuvant dabrafenib/trametinib or placebo in stage 3 melanoma: a correlative analysis of the phase Ill COMBI-AD trial

Methods

Tissue Samples and DNA/RNA Extraction

All patients in the Combi-AD trial had surgical resection of the primary skin tumor and lymph nodes. Tissue samples were submitted for central BRAF testing at the time of screening. If patient's consent allowed, remaining tissue material was used for exploratory biomarker studies.

Sections of 5 (±1) μm thickness were cut from all blocks received. A pathologist visually inspect archival FFPE slides and freshly cut slides from the tumor blocks to identify and notate the approximate percentage of tumor content in the region of interest (ROI) and total tumor area (mm2). Depending on the tumor cell content 4-12 slides were macrodissected and used for DNA/RNA isolation. If the ROI contained less than 10% tumor content, further processing was canceled. DNA/RNA was co-extracted from each baseline or relapse sample available using the Qiagen AIIPrep™ RNA/DNA Extraction from FFPE Tissue Kit.

Targeted Sequencing, Reporting of Tumor Mutation Burden

DNA sequencing was performed using either from the DNA/RNA co-extraction or the remaining DNA from the BRAF testing. DNA was sheared (Covaris ultrasonicator) and underwent end repair, A-tailing, indexed adaptor ligation, and PCR amplification using the TruSeq Nano Library Preparation kit (Illumina). The NGS library is denatured and hybridized to a set of RNA baits designed to bind to fragments from specific genomic regions targeting 570 solid tumor associated genes. The hybrid capture is performed using SureSelect reagents (Agilent). The captured libraries were sequenced on an Illumina HiSeq achieving a mean target coverage of 550× across sequenced libraries.

The resulting sequencing data was processed as follows: First, sequence reads were aligned to the reference human genome (build hg19) using the Burrows-Wheeler Aligner (BWA-MEM) [Li H. and Durbin R., Bioinformatics, 2009] to create a BAM file. Next, the initial BAM file was cleaned with Picard to mark PCR duplicates and the percent of duplicated reads noted [http://picard.sourceforge.net]. Then, Genome Analysis ToolKit was used for local realignment and base quality score recalibration [McKenna et al., Genome Research, 2010; DePristo M. et al., Nature Genetics, 2011]. Single nucleotide variants were identified with MUTECT. Copy number variants were identified using PureCN [Riester et al. Source Code for Bio Med 2016]. Indels were identified using PINDEL [Kai Ye et al., Bioinformatics 2009]. Tumor mutation burden was estimated in targeted protein coding sequence using PureCN [Riester et al. 2016 Source Code for Biology and Medicine]. Briefly, the total number of predicted somatic SNVs detected (including silent mutations) with a minimum allelic fraction of 0.03 in the sequenced coding regions (1.5 Mb) were divided by the total megabases of sequenced coding bases.

Sequencing libraries were included in downstream analyses if mean coverage was at least 100×, had GC and AT dropouts of less than 20%, and there was evidence of tumor content in the sequenced sample (inferred tumor purity >0). Potential sequencing artifacts and germline genetic variants were removed from downstream analyses. Likely artifactual SNVs were identified by low coverage (<50×), low read support (<5 reads supporting the mutant allele), low allelic fraction (<0.01 unless known or likely oncogenic mutation), low average base quality (<25 unless known hotspot mutation), or high proportion of reads with poorly supported alignments (>10% MQO). Likely artifactual indels were identified by low coverage (<50×), low read support (<4), low allelic fraction (<0.04), or overlap with repetitive regions of the genome. Likely germline SNVs and indels were identified by presence in Exome Sequencing Project (http://evs.gs.washington.edu/EVS/) and Exome Aggregation Consortium databases (http://exac.broadinstitute.org/) at appreciable frequency (ESP MAF>0.001 or ExAC count of >3 unless known hotspot mutation). SNVs and indels were assigned a functional significance based on presence in the Catalog of Somatic Mutations in Cancer and functional effect with mutations reported in COSMIC in 5 or more tumors considered as ‘known’ oncogenic, mutations with COSMIC count less than 5 but predicted to lead to a premature truncation of the protein considered as ‘likely’ oncogenic, and all others considered ‘unknown’ oncogenic status. CNVs were considered amplifications if the estimated copy number was at least 7, and CNVs were considered as homozygous deletions if the estimated copy number was 0.5 or less.

Gene Expression Profiling: Filtering/Processing, Normalization

A customized Nanostring gene panel (n=800, 780 genes of interest and 20 housekeeping genes) was used to analyze single genes of interest and gene expression signatures.

Filtering was done on raw, non-normalized data, using the negative controls (to calculate baseline as average+2 SD) and the housekeeping genes to determine quality relative to that baseline. A sample having 11 or more (i.e. more than half) transformed values falling below log 10 (baseline+1)+0.25 was excluded. Adding 1.0 to every value prior to applying logarithms prevents zeroes from appearing as outliers. The 0.25 above baseline ensures that the housekeeping gene expression has sufficient dynamic range to generate a reliable normalization factor.

For the normalization 20 housekeeping genes were used. All pre-defined housekeeping genes were found to have moderate/high mean expression levels and low SD compared to all other genes on the Nanostring panel. The housekeeping genes also showed no significantly differences in expression levels between skin and lymph node samples. The normalization is implemented by multiplying a factor calculated from the geometric mean of HKG's, adjusted by the average normalization factor.
Signal transformed by log(x+1)
Yj=Geometric mean of normalizers per sample
Normalization factor=Avg(Yj)/Yj
Baseline is mean(neg ctrl)+2 sd(neg ctrl). This is calculated independently on each sample, on untransformed data (raw, non-normalized data, using the negative controls).
Log 10(x+1) is applied to baseline and HKG values to evaluate.

Prior selected gene expression signatures were calculated as average normalized gene-expression values for genes included in the signature. Data from all genes were weighted equally in signature calculation; there were no negatively-signed genes in the signatures used.

The majority of the gene expression data was generated using samples from lymph node (60%) and primary skin (35%). As expected, some genes of interest and gene signatures were found to be differentially expressed between skin and lymph node samples (e.g. B-cell genes with higher expression in lymph node). As primary skin samples were more often submitted for patients with stage 3a disease (and vice versa lymph node for stage 3c), tissue source was associated with stage and consequently with relapse-free survival. Therefore, all statistical analyses were run on all samples and lymph node samples alone to make sure potential associations with clinical outcome are not based on the tissue sample collected.

Statistical Analysis

Univariate Cox proportional hazard models were fitted to assess or rank the importance of biomarkers on clinical response. Kaplan-Meier nonparametric survival function estimates were fitted and plotted to visualize the survival characteristics of specific subgroups.

To assess the relationship between two or more predictor variables (clinical variables or biomarker variables), initial exploratory semiparametric hazard regression models were fitted using the method of Kooperberg (1995), as implemented in the R “polspline” package. These exploratory models were examined to identify clinical variables that should be retained in a biomarker analysis; the presence of non-proportional hazard; and interactions among variables. Subsequently Cox proportional hazards models were fitted to understand hazard ratios and make statistical inference.

The statistical significance of specific variables in Cox models was assessed by likelihood ratio tests in which one or more terms are deleted from a model. In some cases tests were based on normal approximations and comparing coefficient estimates to their standard errors.

A standard set of clinical or sample variables were included in most analyses aiming to assess relationship of biomarkers to clinical response:
Baseline tissue source: {Lymph Node/Primary/Metastatic/In Transit Metastases/Missing}
Stage: {IIIA/IIIB/IIIC/Missing}
Metastasis: {Macrometastasis/Micrometastasis/Missing}
BRAF mutation: {V600E/V600K/V600E & V600K}
Number of lymph nodes: Integer
Ulceration: {Yes/No/Missing}
Age: Integer
Sex: {Male/Female}

Several clinical or sample-characterization variables included some missing values. When such a variable was to be included in a model, missing values were imputed using the R “missForest” package.

Results

Patient Characteristics in the Biomarker Cohorts

Evaluable data for DNA-seq and gene expression were obtained from 368 and 507 patients, respectively. The biomarker cohorts represented 58% (RNA), 42% (DNA) of the Combi-AD trial population. Paired DNA-seq and gene expression data (RNA) were available for 301 patients. Baseline characteristics of the biomarker cohorts (DNA-seq, RNA) were compared with the full ITT population. Demographic and baseline clinical characteristics were similar between the biomarker cohorts and trial population for most variables examined. Slight imbalances were observed for stage between the DNA-seq cohort and the ITT population. In particular the 95% Cl for stage IIIA did not overlap between ITT set and DNA-seq set and there were also more events in the D+T arm in the DNA-seq population. This was not observed for the RNA cohort (n=507) and paired DNA-seq/RNA biomarker cohort (n=301).

Genomic Landscape in Early Stage 3 Melanoma at Baseline; Association with Clinical Outcome and Response to Therapy in the Combi-AD Trial

BRAF V600E/K mutations were detected by targeted sequencing in almost all (367/368) baseline samples with available sequencing data, consistent with the results generated by the qPCR based assay (FDA approved Biomerieux BRAF ThxID assay) at the time of the screening. The genomic landscape was as expected for melanoma: the most common non-BRAF V600 genetic aberrations among others were CDKN2A with 27% (including patients with larger deletions encompassing CDKN2B), PTEN in 16%, TP53 in 16%, and ARID2 9%.

The Prognostic Value of Immune Gene Expression Signatures in Untreated and Treated Patients with Stage 3 Melanoma

To identify prognostic genes in stage 3 melanoma, all 780 genes of interest (Nanostring customized panel, see materials and methods) were assessed for their association with RFS in 256 samples from the placebo arm.

Among the top genes which were associated with good clinical outcome were multiple immune markers including T-cell, NK, IFN-γ specific genes. Exploring pre-defined T-cell and IFN-γ specific gene expression signatures demonstrated that tumors with high CD8/T-cell and IFN-γ levels showed a significantly improved relapse-free survival in the placebo arm Overall, immune signatures (e.g. CD8/T-cell, IFN-γ) were among the top prognostic markers in the placebo and treatment arms in the Combi-AD trial.

Interaction Between Tumor Mutation Burden and Immune Gene Expression Signatures in the Placebo and Treatment Arm

Tumor mutation burden was calculated for each tumor sample. Median tumor mutational burden (TMB) was 7.3 SNVs/Mb in baseline tumors. As expected, tumors with BRAF V600K mutation had significantly higher TMB levels (Wilcoxon rank sum test p-value 5e-9;) No strong associations were identified for TMB and other clinical variables (data not shown). In line with previously described findings in lung cancer, we did not find any strong correlation between tumor mutation burden and PD-L1 expression levels, IFN-γ signature or CD8/T-cell immune gene expression signature in the 301 samples with paired DNA-seq and RNA data Although IFN-γ signature and TMB levels are not strongly correlated, both parameters contain significant independent prognostic information in the placebo arm, consequently patients with high-TMB (defined here as the top third) and high IFN-γ signature levels (defined here by median) had a very good clinical outcome (>60% RFS) with surgical resection alone, whereas almost 80% of all patients with low-TMB and low IFN-γ immune signature had a relapse event in the available follow-up period Interestingly, the prognostic association with clinical outcome is maintained in the treatment arm for CD8/T-cell signatures, but considerably weakened for TMB. In fact, a completely different outcome pattern was observed for the TMB-IFN-γ subgroups in the treatment arm: low TMB was initially associated with early (on-treatment) relapse events. However, several relapse events occurred in the TMB-high group after treatment discontinuation (>12 months therapy), especially in tumors with low CD8/T-cell signature levels. The IFN-γ signature and other immune gene expression signatures seem to be important for predicting late relapse events and especially for the TMB-high subgroup high IFN-γ levels seem to tip the balance in favor of an improved long term outcome.

While this analysis is not powered to assess treatment interactions in small biomarker subgroups of interest, the data suggest that patients with low TMB seem to have more pronounced long term benefit from targeted therapy compared to patients with very high TMB, especially if immune gene expression signatures are not detectable. Patients with very high TMB have initially good response to targeted therapy, but then seem to show acquired resistance/progression on targeted therapy if the high numbers of neoantigens cannot be recognized by the immune system (in particular in presence of low IFN-γ levels)

Interaction between TMB and immune gene expression signatures in the treatment arm: The reason for this interaction is not immediately clear as the host-tumor interaction represents a complex, layered interplay of competing factors:
High TMB=genomic instability gives rise to important acquired resistance mutations, which facilitate tumor progression
At the same time targeted therapy leads to cell death and antigen presentation and high mutation levels generate neoepitopes that may be recognized by the immune system. Therefore, high TMB also may lead to an increased likelihood that one of the nonsynonymous mutations will ultimately be uniquely immunogenic. This may be the reason why patients with high TMB have a relatively low relapse rate while on therapy and then after treatment discontinuation, patients with high TMB and low IFN-γ levels show a lot of relapse events after 12 months.
CD8/T-cell and IFNy at the time of harvest may be considered as demonstrating evidence of the host immune system's ability to exert some degree of control over tumor growth. In the case of TMB-high tumors this may tip the balance into two directions
   If no immune cells are present, escape mechanism to targeted therapy are dominant. Lots of heterogeneity for relapse mechanism but little immune infiltrates to control tumor growth. The well-known immune modulatory mechanism of Dabrafenib and Trametinib may be the reason why patients do not relapse on therapy, but it is not a sustainable immune response,
   If CD8/T-cell and IFNy are present=recognition of tumor by immune system dominant, sustainable long term adaptive immune response

1-49. (canceled)

50: A method of identifying a melanoma patient who may be responsive to targeted therapy comprising a BRAF inhibitor, a MEK inhibitor, or a BRAF inhibitor and a MEK inhibitor, the method comprising
a. obtaining a biological sample from the patient,
b. determining a tumor mutation burden (TMB) score from the biological sample, wherein the TMB score of the patent that is at or below a reference TMB score identifies the patient as one who may be responsive to targeted therapy comprising a BRAF inhibitor, MEK inhibitor, or a BRAF inhibitor and a MEK inhibitor.

51: The method of claim 50, wherein the melanoma is a BRAF V600 mutant melanoma.

52: The method of claim 50, wherein the targeted therapy is dabrafenib and trametinib or vemurafenib and cobimetinib.

53: The method of claim 50, wherein the TMB score that is at or below the reference TMB score is 5 mutations/Mb or less, 6 mutations/Mb or less, 7 mutations/Mb or less, 8 mutations/Mb or less, 9 mutations/Mb or less, 10 mutations/Mb or less, 11 mutations/Mb or less, 12 mutations/Mb or less, 13 mutations/Mb or less, 14 mutations/Mb or less, 15 mutations/Mb or less, or 16 mutations/Mb or less.

54: A method of treating a melanoma patient, the method comprising:
a. obtaining a biological sample from the patient,
b. determining a tumor mutation burden (TMB) score from the biological sample,
c. administering an effective amount of targeted therapy comprising a BRAF inhibitor, a MEK inhibitor, or a BRAF inhibitor and a MEK inhibitor to the patient, wherein the TMB score of the patient is at or below a reference TMB score, or
d. administering a combination of an effective amount of targeted therapy comprising a BRAF inhibitor, a MEK inhibitor, or a BRAF inhibitor and a MEK inhibitor, with an effective amount of immuno-oncology therapy comprising a PD-1 or a PD-L1 binding antagonist to the patient, wherein the TMB score of the patient is above a reference TMB score.

55: The method of claim 54, wherein the melanoma is a BRAF V600 mutant melanoma.

56: The method of claim 54, wherein the targeted therapy is dabrafenib and trametinib or vemurafenib and cobimetinib.

57: The method of claim 54, wherein the TMB score that is at or below the reference TMB score is 5 mutations/Mb or less, 6 mutations/Mb or less, 7 mutations/Mb or less, 8 mutations/Mb or less, 9 mutations/Mb or less, 10 mutations/Mb or less, 11 mutations/Mb or less, 12 mutations/Mb or less, 13 mutations/Mb or less, 14 mutations/Mb or less, 15 mutations/Mb or less, or 16 mutations/Mb or less.

58: The method of claim 54, wherein the TMB score that is above the reference TMB score is more than 5 mutations/Mb, more than 6 mutations/Mb, more than 7 mutations/Mb, more than 8 mutations/Mb, more than 9 mutations/Mb, more than 10 mutations/Mb, more than 11 mutations/Mb, more than 12 mutations/Mb, more than 13 mutations/Mb, more than 14 mutations/Mb, more than 15 mutations/Mb, or more than 16 mutations/Mb.

59: A method of identifying a melanoma patient who may be responsive from a targeted therapy comprising a BRAF inhibitor, a MEK inhibitor, or a BRAF inhibitor and a MEK inhibitor; the method comprising
a. obtaining a biological sample from the patient and
b. determining i) a tumor mutation burden (TMB) score and ii) an immune activation score from the biological sample, wherein the TMB score of the patient that is above a reference TMB score and ii) an immune activation score of the patient that is above a reference immune activation score identifies the patient as one who may be responsive from a targeted therapy comprising a BRAF inhibitor, MEK inhibitor, or a BRAF inhibitor and a MEK inhibitor.

60: The method of claim 59, wherein the melanoma is a BRAF V600 mutant melanoma.

61: The method of claim 59, wherein the targeted therapy is dabrafenib and trametinib or vemurafenib and cobimetinib.

62: The method of claim 59, wherein the TMB score that is at or below the reference TMB score is 5 mutations/Mb or less, 6 mutations/Mb or less, 7 mutations/Mb or less, 8 mutations/Mb or less, 9 mutations/Mb or less, 10 mutations/Mb or less, 11 mutations/Mb or less, 12 mutations/Mb or less, 13 mutations/Mb or less, 14 mutations/Mb or less, 15 mutations/Mb or less, or 16 mutations/Mb or less.

63: The method of claim 59, wherein the TMB score that is above the reference TMB score is more than 5 mutations/Mb, more than 6 mutations/Mb, more than 7 mutations/Mb, more than 8 mutations/Mb, more than 9 mutations/Mb, more than 10 mutations/Mb, more than 11 mutations/Mb, more than 12 mutations/Mb, more than 13 mutations/Mb, more than 14 mutations/Mb, more than 15 mutations/Mb, or more than 16 mutations/Mb.

64: The method of claim 59, wherein the immune activation score is assessed by measuring tumor infiltrating lymphocytes, PD-L1, CD8, IFNy, or T-cell inflamed gene expression signatures.

65: A method of treating a melanoma patient, the method comprising:
a. obtaining a biological sample from the patient,
b. determining i) a tumor mutation burden (TMB) score and ii) an immune activation score from the biological sample,
c. administering an effective amount of targeted therapy comprising a BRAF inhibitor, a MEK inhibitor, or a BRAF inhibitor and a MEK inhibitor to the patient, wherein i) the TMB score of the patient is at or below a reference TMB score and ii) the immune activation score of the patient is at or below a reference immune activation score, or
d. administering an effective amount of targeted therapy comprising a BRAF inhibitor, a MEK inhibitor, or a BRAF inhibitor and a MEK inhibitor to the patient, wherein i) the TMB score of the patient is above a reference TMB score and ii) the immune activation score of the patient is above a reference immune activation score, or
e. administering an effective amount of immuno-oncology therapy comprising a PD-1 or a PD-L1 binding antagonist to the patient, wherein i) the TMB score of the patient is above a reference TMB score and ii) the immune activation score of the patient is at or below a reference immune activation score, or
f. administering a combination of an effective amount of targeted therapy comprising a BRAF inhibitor, a MEK inhibitor, or a BRAF inhibitor and a MEK inhibitor, with an effective amount of immuno-oncology therapy comprising a PD-1 or a PD-L1 binding antagonist to the patient, wherein the TMB score of the patient is at or below a reference TMB score and the immune activation score of the patient is above a reference immune activation score.

66: The method of claim 65, wherein the melanoma is a BRAF V600 mutant melanoma.

67: The method of claim 65, wherein the targeted therapy is Dabrafenib and Trametinib or Vemurafenib and Cobimetinib.

68: The method of claim 65, wherein the TMB score that is at or below the reference TMB score is 5 mutations/Mb or less, 6 mutations/Mb or less, 7 mutations/Mb or less, 8 mutations/Mb or less, 9 mutations/Mb or less, 10 mutations/Mb or less, 11 mutations/Mb or less, 12 mutations/Mb or less, 13 mutations/Mb or less, 14 mutations/Mb or less, 15 mutations/Mb or less, or 16 mutations/Mb or less.

69: The method of claim 65, wherein the TMB score that is above the reference TMB score is more than 5 mutations/Mb, more than 6 mutations/Mb, more than 7 mutations/Mb, more than 8 mutations/Mb, more than 9 mutations/Mb, more than 10 mutations/Mb, more than 11 mutations/Mb, more than 12 mutations/Mb, more than 13 mutations/Mb, more than 14 mutations/Mb, more than 15 mutations/Mb, or more than 16 mutations/Mb.

70: The method of claim 65, wherein the immune activation levels are assessed by measuring tumor infiltrating lymphocytes, PD-L1, CD8, IFNy, or T-cell inflamed gene expression signatures.
